(19) United States
(12) Patent Application Publication
(10) Pub. No.: US 2026/0351294 A1
(43) Pub. Date: Nov. 5, 2026

(54) STACKED MEMORY DEVICE AND METHODS FOR MANUFACTURING THE SAME

(71) Applicant: Taiwan Semiconductor Manufacturing Company, Ltd., Hsinchu (TW)

(72) Inventors: Yi-Ching Liu, Hsinchu (TW); Je-Min Hung, Hsinchu (TW)

(73) Assignee: Taiwan Semiconductor Manufacturing Company, Ltd., Hsinchu (TW)

(21) Appl. No.: 19/721,047

(22) Filed: Jun. 26, 2026

Related U.S. Application Data
(60) Provisional application No. 61/027,672, filed on Feb. 11, 2008.

(51) Int. Cl. G11C 5/06; G11C 11/4096; H10B 80/00

(52) U.S. Cl. CPC G11C 5/063; G11C 11/4096; H10B 80/00

(57) ABSTRACT

A device includes a plurality of control transistors formed along a major surface of a single substrate. The device includes a plurality of memory layers respectively formed in a plurality of metallization layers that are disposed over the major surface. Each of the memory layers includes a first memory array comprising a plurality of first memory cells. Each of the memory layers includes a second memory array comprising a plurality of second memory cells. Each of the memory layers includes a third memory cell operatively coupling the plurality of control transistors to the plurality of first memory cells. Each of the memory layers includes a fourth memory cell operatively coupling the plurality of control transistors to the plurality of second memory cells.

### CROSS-REFERENCE TO RELATED APPLICATION

This application claims priority to and the benefit of U.S. Provisional Application No. 64/027,672, filed Apr. 3, 2026, which is incorporated herein by reference in its entirety for all purposes.

### BACKGROUND

The semiconductor industry has experienced rapid growth due to continuous improvements in the integration density of a variety of electronic components (e.g., transistors, diodes, resistors, capacitors, etc.). For the most part, this improvement in integration density has come from repeated reductions in minimum feature size, which allows more components to be integrated into a given area.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 illustrates a perspective view of a stacked memory device including plural memory layers over a base die, in accordance with some embodiments.

FIG. 2 illustrates an example circuit diagram of a portion of the memory layers of FIG. 1, in accordance with some embodiments.

FIG. 3 illustrates performing write operations on a bottom array and a top array of the memory layer of FIG. 2, respectively, in accordance with some embodiments.

FIG. 4 illustrates waveforms of various signals when performing the write operations illustrated in FIG. 3, in accordance with some embodiments.

FIG. 5 illustrates performing read operations on a bottom array and a top array of the memory layer of FIG. 2, respectively, in accordance with some embodiments.

FIG. 6 illustrates waveforms of various signals when performing the read operations illustrated in FIG. 5, in accordance with some embodiments.

FIG. 7 illustrates a perspective view of a stacked memory device including plural memory layers over a base die, in accordance with some embodiments.

FIG. 8 illustrates an example circuit diagram of a portion of the memory layers of FIG. 7, in accordance with some embodiments.

FIG. 9 illustrates performing write operations on a bottom array and a top array of the memory layer of FIG. 8, respectively, in accordance with some embodiments.

FIG. 10 illustrates waveforms of various signals when performing the write operations illustrated in FIG. 9, in accordance with some embodiments.

FIG. 11 illustrates performing read operations on a bottom array and a top array of the memory layer of FIG. 8, respectively, in accordance with some embodiments.

FIG. 12 illustrates waveforms of various signals when performing the read operations illustrated in FIG. 11, in accordance with some embodiments.

FIG. 13 illustrates a perspective view of a stacked memory device including plural memory layers over a base die, in accordance with some embodiments.

FIG. 14 illustrates an example circuit diagram of a portion of the memory layers of FIG. 13, in accordance with some embodiments.

FIG. 15 illustrates performing write operations on a bottom array and a top array of the memory layer of FIG. 14, respectively, in accordance with some embodiments.

FIG. 16 illustrates waveforms of various signals when performing the write operations illustrated in FIG. 15, in accordance with some embodiments.

FIG. 17 illustrates performing read operations on a bottom array and a top array of the memory layer of FIG. 14, respectively, in accordance with some embodiments.

FIG. 18 illustrates waveforms of various signals when performing the read operations illustrated in FIG. 17, in accordance with some embodiments.

FIG. 19 illustrates a perspective view of a stacked memory device including plural memory layers over a base die, in accordance with some embodiments.

FIG. 20 illustrates an example circuit diagram of a portion of the memory layers of FIG. 19, in accordance with some embodiments.

FIG. 21 illustrates performing a write operation on a bottom array of the memory layer of FIG. 20, in accordance with some embodiments.

FIG. 22 illustrates waveforms of various signals when performing the write operation illustrated in FIG. 21, in accordance with some embodiments.

FIG. 23 illustrates performing a read operation on a top array of the memory layer of FIG. 20, in accordance with some embodiments.

FIG. 24 illustrates waveforms of various signals when performing the write operation illustrated in FIG. 23, in accordance with some embodiments.

FIG. 25 illustrates performing a read operation on a bottom array of the memory layer of FIG. 20, in accordance with some embodiments.

FIG. 26 illustrates waveforms of various signals when performing the read operation illustrated in FIG. 25, in accordance with some embodiments.

FIG. 27 illustrates performing a read operation on a top array of the memory layer of FIG. 20, in accordance with some embodiments.

FIG. 28 illustrates waveforms of various signals when performing the read operation illustrated in FIG. 27, in accordance with some embodiments.

FIG. 29 illustrates a perspective view of a stacked memory device including plural memory layers over a base die and an example circuit diagram of a portion of the memory layer, in accordance with some embodiments.

FIG. 30 illustrates a flow chart of a method for fabricating a stacked memory device including plural memory layers over a base die, in accordance with some embodiments.

FIG. 31 illustrates a flow chart of a method for operating a stacked memory device, in accordance with some embodiments.

FIG. 32 illustrates a flow chart of a method for operating a stacked memory device, in accordance with some embodiments.

FIG. 33 illustrates a flow chart of a method for operating a stacked memory device, in accordance with some embodiments.

FIG. 34 illustrates a flow chart of a method for operating a stacked memory device, in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over, or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” “top,” “bottom” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

This disclosure relates to techniques for reducing parasitic loading on bit lines in three-dimensional (3D) stackable memory devices to improve signal margin during read and write operations. Memory devices can be used in a variety of applications, such as data storage, computing systems, and embedded electronic systems. To increase storage density, memory arrays can be stacked vertically over a base die, such that multiple memory layers can share peripheral circuitry including control, driver, and input/output (IO) circuitry disposed in the base die. A stacked memory array can include local bit lines that couple memory cells along a column to a global bit line, and the global bit line can route read and write signals between the stacked memory arrays and the base die.

As the number of word lines along a bit line increases to raise storage density, the length of the bit line can also increase, and the parasitic resistance and parasitic capacitance on the bit line can grow accordingly. The increased parasitic resistance and parasitic capacitance on the local bit line can degrade the signal margin during a signal developing phase of read and write operations. A longer bit line can reduce the sensing margin available to distinguish stored logic states. A longer bit line can also reduce read and write performance, such that read speed and write speed can be slowed. These drawbacks can limit the scalability of stacked memory arrays having a large number of word lines per bit line.

The techniques described herein can provide a stacked memory architecture in which a far-side bit line is promoted to a higher metallization layer as a fly bit line to reduce the parasitic resistance and parasitic capacitance presented on the local bit line. The techniques described herein can apply such promotion to a read bit line, a write bit line, or both, such that the bit line associated with an array that is far from a global bit line can be routed through a higher metallization layer rather than through the local bit line path. By routing the far-side bit line through a higher metallization layer, the techniques described herein can reduce the local bit line loading seen during signal development and can improve the signal margin during read and write operations.

To implement the techniques described herein, a memory device can include a base die and a number of memory arrays stacked over the base die. Each of the stacked memory arrays (sometimes referred to as a memory layer) can be formed across one or more metallization layers over the base die. Such a stacked memory architecture is sometimes referred to as a monolithic memory device. Further, each of the stacked memory arrays can be divided into two or more groups along a lateral direction, in accordance with some embodiments. For example, each of the stacked memory arrays can include a top array and a bottom array disposed with respect to each other along the lengthwise direction of bit lines. The top array and the bottom array can each include a memory cell region and a gain cell region, such as, the bottom array including a bottom memory cell region and a bottom gain cell region and the top array including a top memory cell region and a top gain cell region. The top/bottom memory cell region includes a number of memory cells each configured to store a data bit, and the top/bottom gain cell region includes a number of gain cells each configured to provide a current output representative of a stored logic state.

Both of the top array and bottom array (of all the stacked memory arrays) can be coupled to the base die through a number of global bit lines vertically extending across those stacked memory arrays. These global bit lines can be disposed on a side of the memory arrays along the lengthwise direction of the bit lines. In some embodiments, a far-side bit line associated with the array (e.g., the top array) that is farther from the global bit line can be routed as a fly bit line on a higher metallization layer, while a near-side bit line associated with the array (e.g., the bottom array) that is closer to the global bit line can be routed through a local bit line path. The stacked memory architecture can be extended to more than two groups, such that one or more of the memory arrays can each be divided into n groups with multiple fly bit lines routing far-side read and write paths across the groups.

The techniques described herein can support a range of operational modes for the stacked memory architecture. For example, separate top and bottom write bit lines and separate top and bottom read bit lines can support parallel write and parallel read of two bits at a time. As another example, a shared write bit line can support sequential write to the top and bottom arrays under control of write enable signals, while separate top and bottom read bit lines can support parallel read. As a further example, ratioed gain cells having different sizes can be used on the top and bottom read paths, such that the currents from the top read bit line and the bottom read bit line can be merged on the global bit line to form a multi-level current that represents a two-bit combination. As yet another example, a single write bit line and a single read bit line can support one-bit write and one-bit read, with selection between the top and bottom arrays controlled by top and bottom read word line signals.

By promoting the far-side read and write bit lines to a higher metallization layer, the techniques described herein can reduce the parasitic resistance and parasitic capacitance presented on the local bit line during signal developing phases. The reduced parasitic loading can provide improved signal margin during read and write operations and can support faster read and write speeds relative to approaches that route both the near-side and far-side bit lines through the local bit line path. When the memory array is divided into n groups, the parasitic resistance and parasitic capacitance on the local bit line can be reduced to approximately 1/n times the value associated with an undivided bit line of the same overall length. The techniques described herein can therefore support stacked memory architectures having a larger number of word lines per bit line, such as architectures with 8 stacks or 16 stacks, while preserving signal margin and read and write performance.

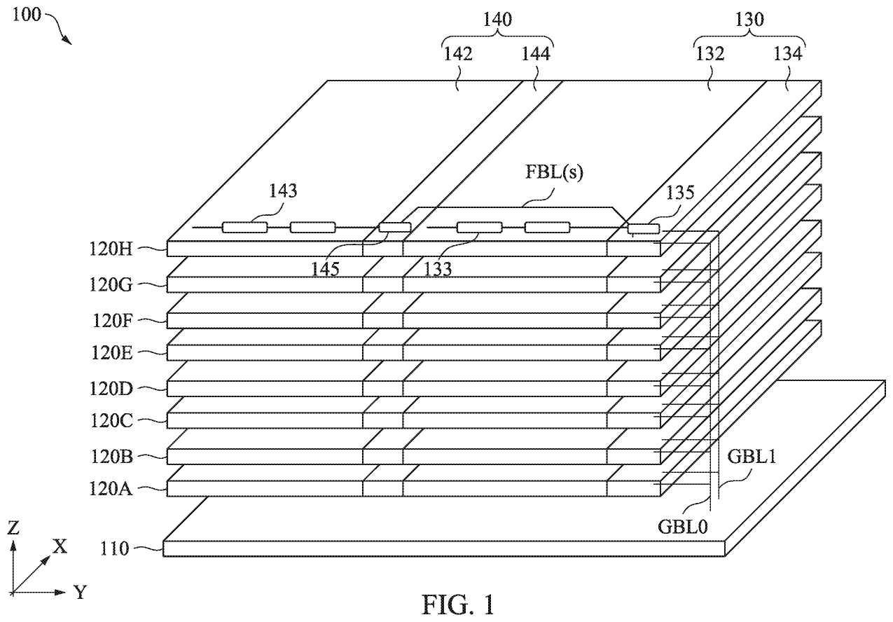

Referring to FIG. 1, a memory device 100 can include a base die 110 and a plurality of memory layers such as, for example, memory layer 120A, memory layer 120B, memory layer 120C, memory layer 120D, memory layer 120E, memory layer 120F, memory layer 120G, and memory layer 120H stacked over the base die 110 along a vertical direction. The base die 110 can include control, driver, and input/output (I/O) circuitry (collectively referred to as peripheral circuitry) that facilitates read and write operations on the memory layer 120A through memory layer 120H. In some implementations, the memory layer 120A through memory layer 120H can be respectively formed in a plurality of metallization layers disposed over a major surface of the base die 110, such that the memory layer 120A through memory layer 120H share the peripheral circuitry of the base die 110. For example, the memory device 100 can be arranged as an 8-stack memory, with each of the memory layer 120A through memory layer 120H providing a separate memory plane along the Z direction above the base die 110 over the X-Y plane.

Each of the memory layers 120A through memory layer 120H can include a bottom array 130 and a top array 140 disposed adjacent to each other along a lateral direction parallel to the lengthwise direction of local bit lines. The bottom array 130 can include a bottom memory cell region 132 and a bottom gain cell region 134, and the top array 140 can include a top memory cell region 142 and a top gain cell region 144. In some implementations, the bottom memory cell region 132 can include a plurality of bottom memory cells 133 each operable to store a data bit, and the bottom gain cell region 134 can include a plurality of bottom gain cells 135 each operable to output a current representative of a stored logic state of a corresponding one of the bottom memory cells 133. For example, the top memory cell region 142 can include a plurality of top memory cells 143 arranged along the lateral direction, and the top gain cell region 144 can include a plurality of top gain cells 145 that receive a voltage signal from the top memory cells 143 and output a corresponding current signal. The bottom array 130 and the top array 140 can be disposed such that the top gain cell region 144 is interposed between the top memory cell region 142 and the bottom gain cell region 134 along the lateral direction.

The memory device 100 can include a number of global bit lines such as, for example, GBL0 and GBL1, that vertically extend across the metallization layers in which the memory layer 120A through memory layer 120H are formed. The global bit line GBL0 and the global bit line GBL1 can route read and write signals between the memory layer 120A through memory layer 120H and the base die 110. In some implementations, the global bit line GBL0 and the global bit line GBL1 can be disposed on a side of the memory layer 120A through memory layer 120H along the lateral direction, such that the bottom array 130 is closer to the global bit line GBL0 and the global bit line GBL1 than the top array 140. For example, the bottom array 130 can be a near-side array with respect to the global bit line GBL0 and the global bit line GBL1, and the top array 140 can be a far-side array with respect to the global bit line GBL0 and the global bit line GBL1. In some embodiments, the global bit line GBL0 may be configured to route the signals from the bottom array 130 (of each of the memory layers) to the base die 110, and the global bit line GBL1 may be configured to route the signals from the top array 140 (of each of the memory layers) to the base die 110.

Each of the memory layer 120A through memory layer 120H can include one or more fly bit lines FBL(s) that route a far-side bit line path from the top array 140 across the bottom array 130 to the global bit line GBL1. The fly bit line FBL can be formed in a higher metallization layer than the local bit lines that couple the top memory cells 143 and the bottom memory cells 133 along the lateral direction, such that the parasitic resistance and parasitic capacitance presented on the local bit lines are reduced. In some implementations, the fly bit line FBL can extend laterally over (or fly over) the bottom memory cell region 132 and the bottom gain cell region 134 within a corresponding one of the memory layer 120A through memory layer 120H, and can couple to the global bit line GBL1 that may be formed as a vertical via disposed on the side of the bottom gain cell region 134. In the illustrative example of FIG. 1, the fly bit line FBL can include a fly read bit line and a fly write bit line placed on a metallization layer higher than another metallization layer, where other bit lines and/or word lines are formed, to facilitate far-side read and write operations for the top array 140, while a near-side bit line path couples the bottom array 130 to the global bit line GBL0.

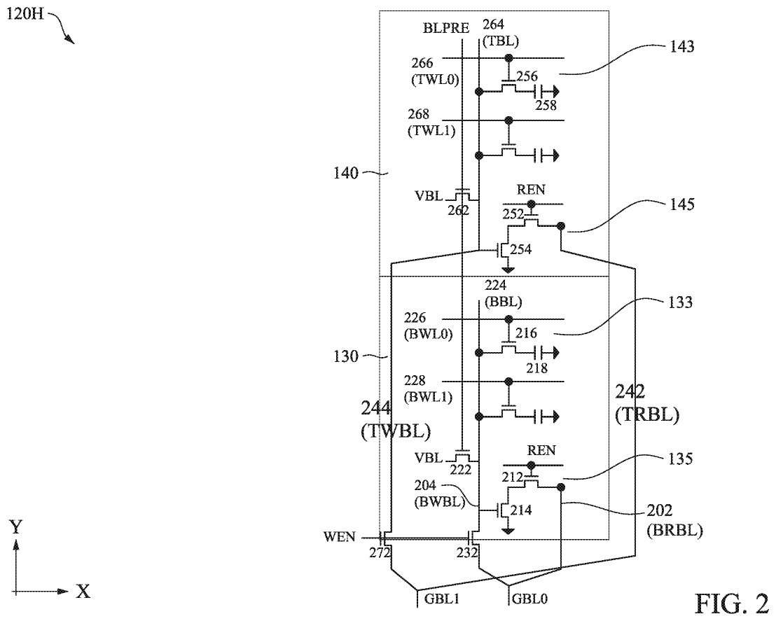

Referring to FIG. 2, a circuit diagram of the memory layer 120H is shown as a representative example of the memory layers described with respect to FIG. 1, in accordance with some embodiments. The memory layer 120H can include the top array 140 and the bottom array 130 disposed adjacent to each other along a lateral direction (e.g., the Y direction), with the top array 140 being a far-side array relative to the global bit line GBL1 and the global bit line GBL0, and the bottom array 130 being a near-side array relative to the global bit line GBL1 and the global bit line GBL0. The top array 140 can include the top memory cell 143 and the top gain cell 145, and the bottom array 130 can include the bottom memory cell 133 and the bottom gain cell 135. The memory layer 120H can be coupled to the base die through the global bit line GBL 1 and the global bit line GBL0, with the top write bit line 244 and the top read bit line 242 routing far-side write and read paths from the top array 140 across the bottom array 130 as fly bit lines formed on a higher metallization layer.

Referring first to the bottom array 130, the bottom gain cell 135 includes transistors 212 and 214 (e.g., both in n-type) coupled to each other in series. The transistor 212 has a first source/drain terminal coupled to a local bit line 202, sometimes referred to as bottom read bit line (BRBL) 202, and a second source/drain terminal coupled to a first source/drain terminal of the transistor 214. The transistor 214 has a second source/drain terminal coupled to ground. Further, the transistor 212 has a gate terminal coupled to a read enable signal REN, and the transistor 214 has a gate terminal coupled to a local bit line 224, sometimes referred to as bottom bit line (BBL) 224. In some embodiments, the BBL 224 and a bit line voltage (VBL) can be coupled to first and second source/drain terminals of a transistor 222, respectively, and the transistor 222 has a gate terminal configured to receive a bit line pre-charge signal BLPRE. In some embodiments, the BBL 224 is coupled to the global bit line GBL0 through a transistor 232, and the transistor 232 has a gate terminal configured to receive a write enable signal WEN. Referring still to the bottom array 130, the bottom memory cell 133 includes a transistor 216 and a capacitor 218 coupled to each other in series. The transistor 216 has its first and second source/drain terminals coupled to the BBL 224 and one of the terminals of the capacitor 218, respectively. The other terminal of the capacitor 218 is coupled to ground. Further, a gate terminal of the transistor 216 is coupled to a local word line 226, sometimes referred to as bottom word line 0 (BWL0) 226. By asserting the BWL0 226, the transistor 216 can be activated, which allows access to the bottom memory cell 133. Similarly, a gate terminal of another transistor is coupled to a local word line 228, sometimes referred to as bottom word line 1 (BWL1) 228. By asserting the BWL1 228, the corresponding transistor can be activated, which allows access to the bottom memory cell 133.

Referring next to the top array 140, the top gain cell 145 includes transistors 252 and 254 (e.g., both in n-type) coupled to each other in series. The transistor 252 has a first source/drain terminal coupled to a local bit line 242, sometimes referred to as top read bit line (TRBL) 242, and a second source/drain terminal coupled to a first source/drain terminal of the transistor 254. The TRBL 242 is coupled to the global bit line GBL1. The transistor 254 has a second source/drain terminal coupled to ground. Further, the transistor 252 has a gate terminal coupled to the same read enable signal REN (received by the transistor 212 of the bottom gain cell 135), and the transistor 254 has a gate terminal coupled to a local bit line 264, sometimes referred to as top bit line (TBL) 264, and further coupled to a local bit line 244, sometimes referred to as top write bit line (TWBL) 244. In some embodiments, the TBL 264 and the bit line voltage (VBL) can be coupled to first and second source/drain terminals of a transistor 262, respectively, and the transistor 262 has a gate terminal operating according to the bit line pre-charge signal BLPRE.

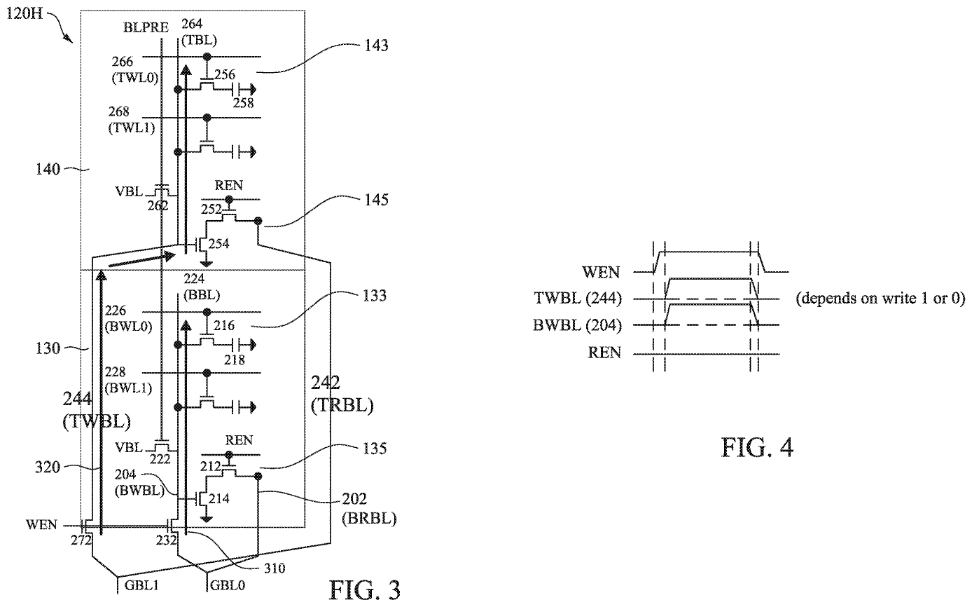

In some embodiments, the TWBL 244 is coupled to the global bit line GBL1 through a transistor 272, and the transistor 272 has a gate terminal operating according to the write enable signal WEN. In some embodiments, the TWBL 244 and TRBL 242 may each be formed as one of the fly bit lines FBLs that are configured to access the top array 140. The TRBL 242 may be configured as a part of a signal path to read the memory cells of the top array 140, and the TWBL 244 may be configured as a part of a signal path to write the memory cells of the top array 140, details of which will be discussed below in FIGS. 3-6. Referring still to the top array 140, the top memory cell 143 includes transistor 256 and capacitor 258 coupled to each other in series. The transistor 256 has its first and second source/drain terminals coupled to the TBL 264 and one of the terminals of the capacitor 258, respectively. The other terminal of the capacitor 258 is coupled to ground. Further, a gate terminal of the transistor 256 is coupled to a local word line 266, sometimes referred to as top word line 0 (TWL0) 266. By asserting the TWL0 266, the transistor 256 can be activated, which allows access to the top memory cell 143. Similarly, a gate terminal of another transistor is coupled to a local word line 268, sometimes referred to as top word line 1 (TWL1) 268. By asserting the TWL1 268, the corresponding transistor can be activated, which allows access to the top memory cell 143.

Referring to FIG. 3, the memory layer 120H is shown as a representative example, with a first signal path, shown as signal path 310, directed to a write operation on the bottom memory cell 133, and a second signal path, shown as signal path 320, directed to a write operation on the top memory cell 143, in accordance with some embodiments. The TWBL 244 routes far-side write paths from the top array 140 across the bottom array 130 as one of the fly bit lines FBLs formed in a higher metallization layer, while other bit lines (e.g., 202, 204), routing near-side write paths from the bottom array 130 to the global bit line GBL0, are formed in a lower metallization layer.

During a write operation directed to the bottom memory cell 133, a data signal provided on the global bit line GBL0 propagates along the signal path 310 through the transistor 232 activated by the write enable signal WEN, onto the BWBL 204 and the BBL 224, and into the bottom memory cell 133 selected by assertion of the BWL0 226. The BWBL 204 and the BBL 224 may be formed as different portion of one local bit line, in some embodiments. In some embodiments, the voltage level developed on the BWBL 204 and the BBL 224 is determined by the data value presented on the global bit line GBL0. For example, when writing a logic high to the bottom memory cell 133, the global bit line GBL0, the BWBL 204, and the BBL 224 represent a logic high voltage level, such that the capacitor 218 of the selected bottom memory cell 133 is charged through the transistor 216 activated by the corresponding BWL0 226.

During a write operation directed to the top memory cell 143, a data signal provided on the global bit line GBL1 propagates along the signal path 320 through the transistor 272 activated by the write enable signal WEN, onto the TWBL 244 and further onto the TBL 264, and into the top memory cell 143 selected by assertion of the TWL0 266. In some embodiments, the voltage level developed on the TWBL 244 and the TBL 264 is determined by the data value presented on the global bit line GBL1. For example, when writing a logic low to the top memory cell 143, the global bit line GBL1, the TWBL 244, and the TBL 264 represent a logic low voltage level, such that the capacitor 258 of the selected top memory cell 143 is discharged through the transistor 256 activated by the corresponding TWL0 266. In some embodiments, the TWBL 244 and the BWBL 204 are electrically decoupled from each other, such that a first data bit may be written to the bottom memory cell 133 along the signal path 310 and a second data bit may be written to the top memory cell 143 along the signal path 320 in parallel.

Referring to FIG. 4, a diagram illustrating waveforms of signals, while writing the bottom array 130 and the top array 140 in parallel through the BWBL 204 and the TWBL 244, is shown, in accordance with some embodiments. For example, the write enable signal WEN, the signals present on the TWBL 244, the signal present on the BWBL 204, and the read enable signal REN are shown. It should be understood that the waveforms of FIG. 4 are provided merely for illustrative purposes, and do not intend to limit the scope of the present disclosure.

During a write operation, the write enable signal WEN transitions from a logic low voltage level (VL) to a logic high voltage level (VH) first, such that the transistor 272 and the transistor 232 are activated to couple the global bit line GBL1 to the TWBL 244 and to couple the global bit line GBL0 to the BWBL 204, respectively. After the write enable signal WEN transitions to VH, the TWBL 244 and the BWBL 204 transition from VL to VH according to respective data values driven onto the global bit line GBL1 and the global bit line GBL0. The read enable signal REN is maintained at VL throughout the write operation, such that the transistor 252 and the transistor 212 remain deactivated, and the TRBL 242 and the BRBL 202 are decoupled from the write current paths through the top gain cell 145 and the bottom gain cell 135. After the write operation is completed, the write enable signal WEN transitions from VH back to VL. In some embodiments, the data values driven onto the TWBL 244 and the BWBL 204 are independent of each other, such that a first bit may be written to the top memory cell 143 along the signal path 320 and a second bit may be written concurrently to the bottom memory cell 133 along the signal path 310. For example, when writing a logic zero to the top memory cell 143 and a logic one to the bottom memory cell 133 in parallel, the global bit line GBL1 and the top write bit line 244 represent VL while the global bit line GBL0 and the bottom write bit line 204 represent VH during the interval in which the write enable signal WEN is at VH.

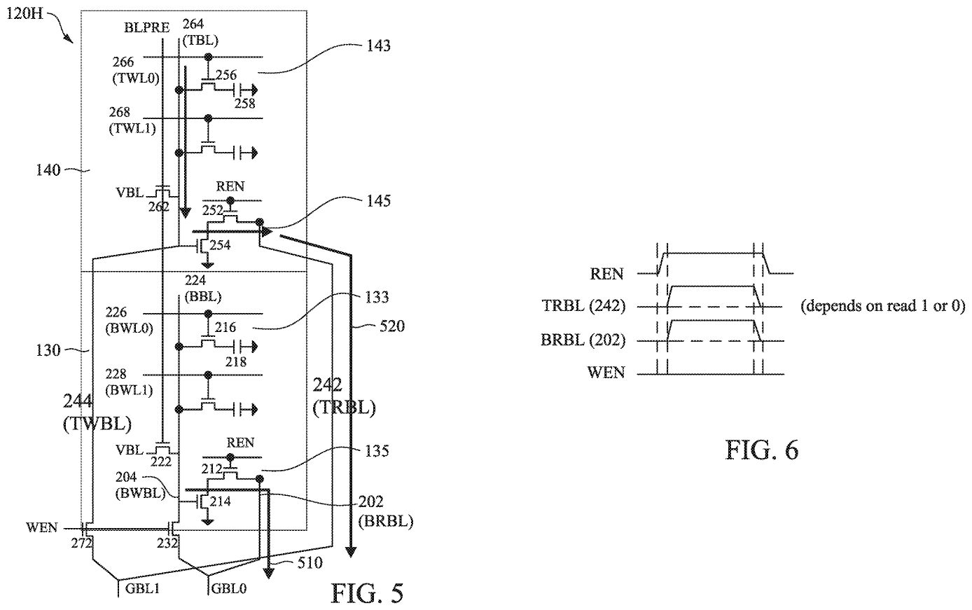

Referring to FIG. 5, the memory layer 120H is shown as a representative example, with a first signal path, shown as signal path 510, directed to a read operation on the bottom memory cell 133, and a second signal path, shown as signal path 520, directed to a read operation on the top memory cell 143, in accordance with some embodiments. The TRBL 242 routes a far-side read path from the top array 140 across the bottom array 130 as one of the FBLs formed in a higher metallization layer, while the BRBL 202, routing a near-side read path from the bottom array 130 to the global bit line GBL0, is formed in a lower metallization layer.

During a read operation directed to the bottom memory cell 133, a data signal stored by the bottom memory cell 133 selected by assertion of the BWL0 226 propagates along the signal path 510 onto the BBL 224 (as a voltage signal), through the transistor 212 activated by the read enable signal REN, and onto the global bit line GBL0 as a current signal generated by the bottom gain cell 135. Stated another way, the bottom gain cell 135 is configured to convert a voltage signal into a current signal. In some embodiments, the current level developed on the BRBL 202 is determined by the data value stored by the selected bottom memory cell 133. For example, when reading a logic low from the bottom memory cell 133, the BRBL 202 and the global bit line GBL0 represent a low current level (IL), and when reading a logic high from the bottom memory cell 133, the BRBL 202 and the global bit line GBL0 represent a high current level (IH).

During a read operation directed to the top memory cell 143, a data signal stored by the top memory cell 143 selected by assertion of the TWL0 266 propagates along the signal path 520 onto the TBL 264, through the transistor 252 activated by the read enable signal REN, and onto the global bit line GBL1 as a current signal generated by the top gain cell 145. Stated another way, the top gain cell 145 is configured to convert a voltage signal into a current signal. In some embodiments, the current level developed on the TRBL 242 is determined by the data value stored by the selected top memory cell 143. For example, when reading a logic low from the top memory cell 143, the TRBL 242 and the global bit line GBL1 represent a low current level (IL), and when reading a logic high from the top memory cell 143, the TRBL 242 and the global bit line GBL1 represent a high current level (IH). In some embodiments, the TRBL 242 and the BRBL 202 are electrically decoupled from each other, such that a first data bit may be read from the bottom memory cell 133 along the signal path 510 and a second data bit may be read from the top memory cell 143 along the signal path 520 in parallel.

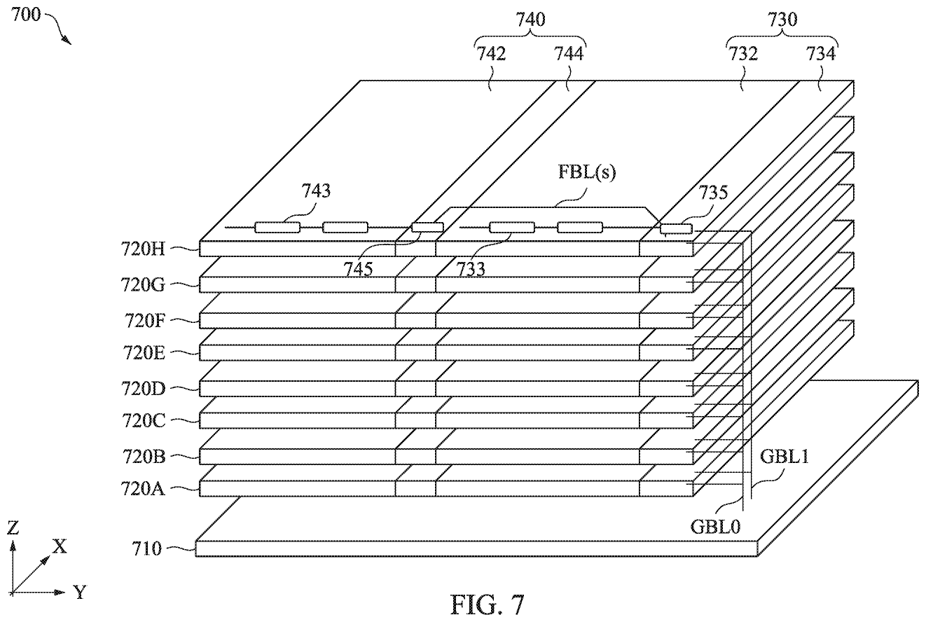

Referring to FIG. 7, a memory device 700 can include a base die 710 and a plurality of memory layers such as, for example, memory layer 720A, memory layer 720B, memory layer 720C, memory layer 720D, memory layer 720E, memory layer 720F, memory layer 720G, and memory layer 720H stacked over the base die 710 along a vertical direction. The base die 710 can include control, driver, and input/output (I/O) circuitry that facilitates read and write operations on the memory layer 720A through memory layer 720H. In some implementations, the memory layer 720A through memory layer 720H can be respectively formed in a plurality of metallization layers disposed over a major surface of the base die 710, such that the memory layer 720A through memory layer 720H share the peripheral circuitry of the base die 710. For example, the memory device 700 can be arranged as an 8-stack memory, with each of the memory layer 720A through memory layer 720H providing a separate memory plane along the Z direction above the base die 710 over the X-Y plane.

Each of the memory layer 720A through memory layer 720H can include a first array, shown as bottom array 730, and a second array, shown as top array 740, disposed adjacent to each other along a lateral direction parallel to the lengthwise direction of local bit lines. The bottom array 730 can include a bottom memory cell region 732 and a bottom gain cell region 734, and the top array 740 can include a top memory cell region 742 and a top gain cell region 744. In some implementations, the bottom memory cell region 732 can include a plurality of bottom memory cells 733 each operable to store a data bit, and the bottom gain cell region 734 can include a plurality of bottom gain cells 735 each operable to output a current representative of a stored logic state of a corresponding one of the bottom memory cells 733. For example, the top memory cell region 742 can include a plurality of top memory cells 743 arranged along the lateral direction, and the top gain cell region 744 can include a plurality of top gain cells 745 that receive a voltage signal from the top memory cells 743 and output a corresponding current signal. The bottom array 730 and the top array 740 can be disposed such that the top gain cell region 744 is interposed between the top memory cell region 742 and the bottom gain cell region 734 along the lateral direction.

The memory device 700 can include a number of global bit lines such as, for example, GBL0 and GBL1, that vertically extend across the metallization layers in which the memory layer 720A through memory layer 720H are formed. The global bit line GBL0 and the global bit line GBL1 can route read and write signals between the memory layer 720A through memory layer 720H and the base die 710. In some implementations, the global bit line GBL0 and the global bit line GBL1 can be disposed on a side of the memory layer 720A through memory layer 720H along the lateral direction, such that the bottom array 730 is closer to the global bit line GBL0 and the global bit line GBL1 than the top array 740. For example, the bottom array 730 can be a near-side array with respect to the global bit line GBL0 and the global bit line GBL1, and the top array 740 can be a far-side array with respect to the global bit line GBL0 and the global bit line GBL1. In some embodiments, the global bit line GBL0 may be configured to route signals associated with the bottom gain cells 735 of each of the memory layers to the base die 710, and the global bit line GBL1 may be configured to route signals associated with the top gain cells 745 of each of the memory layers to the base die 710.

Each of the memory layer 720A through memory layer 720H can include one or more fly bit lines FBL(s) that route a far-side bit line path from the top array 740 across the bottom array 730 to the global bit line GBL1. The fly bit lines FBL(s) can be formed in a higher metallization layer than the local bit lines that couple the top memory cells 743 and the bottom memory cells 733 along the lateral direction, such that the parasitic resistance and parasitic capacitance presented on the local bit lines are reduced. In some implementations, the fly bit lines FBL(s) can extend laterally over the top memory cell region 742 and the top gain cell region 744 within a corresponding one of the memory layer 720A through memory layer 720H, and can couple to the global bit line GBL1 that may be formed as a vertical via disposed on the side of the bottom gain cell region 734. In the illustrative example of FIG. 7, the fly bit lines FBL(s) can include a top read bit line placed on a metallization layer higher than another metallization layer, where other bit lines and/or word lines are formed, to facilitate a far-side read operation for the top array 740, while a near-side bit line path couples the bottom array 730 to the global bit line GBL0, and a shared write bit line supports a one-bit write function selectable between the top array 740 and the bottom array 730 under control of write enable signals generated by the base die 710.

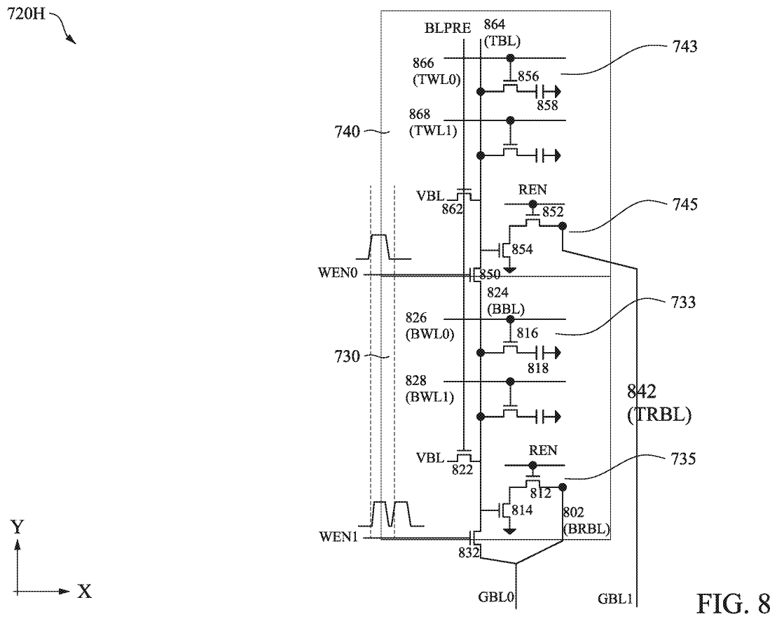

Referring to FIG. 8, a circuit diagram of the memory layer 720H is shown as a representative example of the memory layers described with respect to FIG. 7, in accordance with some embodiments. The memory layer 720H can include the top array 740 and the bottom array 730 disposed adjacent to each other along a lateral direction (e.g., the Y direction), with the top array 740 being a far-side array relative to the global bit line GBL1 and the global bit line GBL0, and the bottom array 730 being a near-side array relative to the global bit line GBL1 and the global bit line GBL0. The top array 740 can include the top memory cell 743 and the top gain cell 745, and the bottom array 730 can include the bottom memory cell 733 and the bottom gain cell 735. The memory layer 720H can be coupled to the base die through the global bit line GBL0 and a top read bit line that routes a far-side read path from the top array 740 across the bottom array 730 as an FBL formed on a higher metallization layer, while a shared write path supports a one-bit write function selectable between the top array 740 and the bottom array 730 under control of write enable signals WEN0 and WEN1 generated by the base die.

Referring first to the bottom array 730, the bottom gain cell 735 includes transistors 812 and 814 (e.g., both in n-type) coupled to each other in series. The transistor 812 has a first source/drain terminal coupled to a local bit line 802, sometimes referred to as bottom read bit line (BRBL) 802, and a second source/drain terminal coupled to a first source/drain terminal of the transistor 814. The transistor 814 has a second source/drain terminal coupled to ground. Further, the transistor 812 has a gate terminal configured to receive a read enable signal REN, and the transistor 814 has a gate terminal coupled to a local bit line 824, sometimes referred to as bottom bit line (BBL) 824. In some embodiments, the BBL 824 and a bit line voltage (VBL) can be coupled to first and second source/drain terminals of a transistor 822, respectively, and the transistor 822 has a gate terminal operating according to a bit line pre-charge signal BLPRE. In some embodiments, the BBL 824 is coupled to the global bit line GBL0 through a transistor 832, and the transistor 832 has a gate terminal operating according to the write enable signal WEN1. Referring still to the bottom array 730, the bottom memory cell 733 includes a transistor 816 and a capacitor 818 coupled to each other in series. The transistor 816 has its first and second source/drain terminals coupled to the BBL 824 and one of the terminals of the capacitor 818, respectively. The other terminal of the capacitor 818 is coupled to ground. Further, a gate terminal of the transistor 816 is coupled to a local word line 826, sometimes referred to as bottom word line 0 (BWL0) 826. By asserting the BWL0 826, the transistor 816 can be activated, which allows access to the bottom memory cell 733. Similarly, a gate terminal of another transistor is coupled to a local word line 828, sometimes referred to as bottom word line 1 (BWL1) 828. By asserting the BWL1 828, the corresponding transistor can be activated, which allows access to the bottom memory cell 733.

Referring next to the top array 740, the top gain cell 745 includes transistors 852 and 854 (e.g., both in n-type) coupled to each other in series. The transistor 852 has a first source/drain terminal coupled to a local bit line 842, sometimes referred to as top read bit line (TRBL) 842, and a second source/drain terminal coupled to a first source/drain terminal of the transistor 854. The TRBL 842, formed as one of the fly bit lines FBL(s), is coupled to the global bit line GBL1 as a dedicated read bit line path for the top array 740. The transistor 854 has a second source/drain terminal coupled to ground. Further, the transistor 852 has a gate terminal configured to receive the read enable signal REN, and the transistor 854 has a gate terminal coupled to a local bit line 864, sometimes referred to as top bit line (TBL) 864. The BBL 824 and the TBL 864 may be formed as different portion of one local bit line, in some embodiments. In some embodiments, the TBL 864 and the bit line voltage (VBL) can be coupled to first and second source/drain terminals of a transistor 862, respectively, and the transistor 862 has a gate terminal operating according to the bit line pre-charge signal BLPRE.

In some embodiments, the TBL 864 is coupled to the BBL 824 through a transistor 850, and the transistor 850 has a gate terminal operating according to the write enable signal WEN0. In some embodiments, the transistor 850 and the transistor 832 are jointly activated to propagate a data signal from the global bit line GBL0 to the TBL 864 during a top bit line write operation, while the transistor 832 alone is activated during a bottom bit line write operation, such that a single write bit line path supports one-bit write function selectable between the top array 740 and the bottom array 730. Referring still to the top array 740, the top memory cell 743 includes a transistor 856 and a capacitor 858 coupled to each other in series. The transistor 856 has its first and second source/drain terminals coupled to the TBL 864 and one of the terminals of the capacitor 858, respectively. The other terminal of the capacitor 858 is coupled to ground. Further, a gate terminal of the transistor 856 is coupled to a local word line 866, sometimes referred to as top word line 0 (TWL0) 866. By asserting the TWL0 866, the transistor 856 can be activated, which allows access to the top memory cell 743. Similarly, a gate terminal of another transistor is coupled to a local word line 868, sometimes referred to as top word line 1 (TWL1) 868. By asserting the TWL1 868, the corresponding transistor can be activated, which allows access to the top memory cell 743.

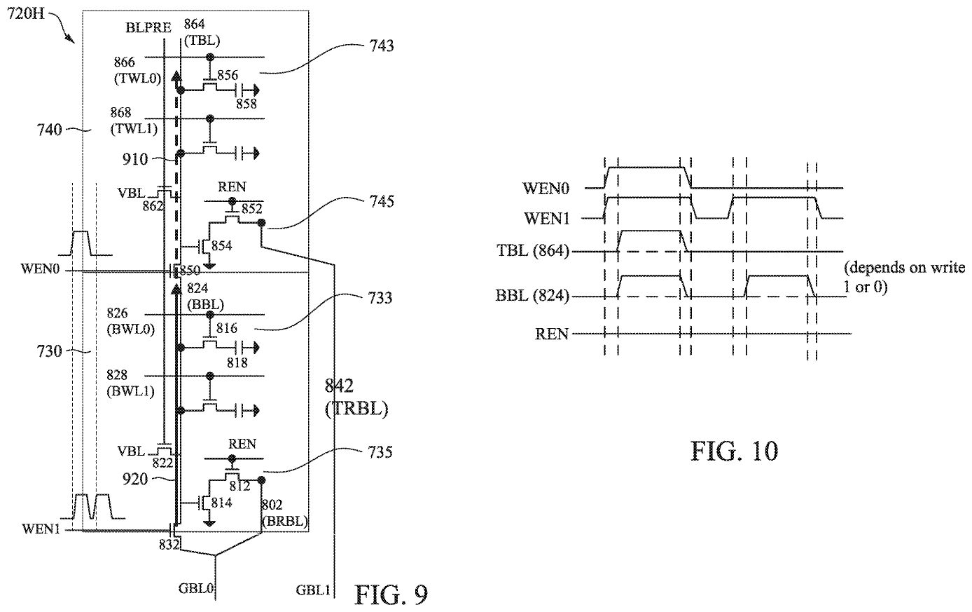

Referring to FIG. 9, the memory layer 720H is shown as a representative example, with a first signal path, shown as signal path 910, directed to a write operation on the top memory cell 743, and a second signal path, shown as signal path 920, directed to a write operation on the bottom memory cell 733, in accordance with some embodiments. In the example of FIG. 9, the write operation performed on the bottom memory cell 733 may be subsequent to the write operation performed on the top memory cell 743. As such, the signal path 910 and the signal path 920 may share the global bit line GBL0 and the BBL 824 as a common write bit line path, such that a one-bit write function is selectable between the top array 740 and the bottom array 730 under control of the write enable signal WEN0 and the write enable signal WEN1 generated by the base die.

During a write operation directed to the top memory cell 743, a data signal provided on the global bit line GBL0 propagates along the signal path 910 through the transistor 832 activated by the write enable signal WEN1, onto the BBL 824, through the transistor 850 activated by the write enable signal WEN0, and further onto the TBL 864, and into the top memory cell 743 selected by assertion of the TWL0 866. In some embodiments, the voltage level developed on the BBL 824 and the TBL 864 is determined by the data value presented on the global bit line GBL0. For example, when writing a logic high to the top memory cell 743, the global bit line GBL0, the BBL 824, and the TBL 864 represent a logic high voltage level, such that the capacitor 858 of the selected top memory cell 743 is charged through the transistor 856 activated by the corresponding TWL0 866.

During a subsequent write operation directed to the bottom memory cell 733, a next data signal provided on the global bit line GBL0 propagates along the signal path 920 through the transistor 832 activated by the write enable signal WEN1, onto the BBL 824, and into the bottom memory cell 733 selected by assertion of the BWL0 826. In some embodiments, the transistor 850 may remain deactivated during the write operation directed to the bottom memory cell 733, such that the TBL 864 is decoupled from the BBL 824 and the top memory cell 743 is not disturbed by the data signal propagating along the signal path 920. For example, when writing a logic low to the bottom memory cell 733, the global bit line GBL0 and the BBL 824 represent a logic low voltage level, such that the capacitor 818 of the selected bottom memory cell 733 is discharged through the transistor 816 activated by the corresponding BWL0 826. In some embodiments, the signal path 910 and the signal path 920 are executed sequentially, such that the top memory cell 743 is written first and the bottom memory cell 733 is written thereafter using the single write bit line path.

Referring to FIG. 10, a diagram illustrating waveforms of signals, while sequentially writing the top array 740 and then the bottom array 730 through the shared write bit line path, is shown, in accordance with some embodiments. For example, the write enable signal WEN0, the write enable signal WEN1, the signals present on the TBL 864, the signals present on the BBL 824, and the read enable signal REN are shown. It should be understood that the waveforms of FIG. 10 are provided merely for illustrative purposes, and do not intend to limit the scope of the present disclosure.

During a sequential write operation, the top memory cell 743 may be written first. Accordingly, both the write enable signal WEN0 and the write enable signal WEN1 transition from a logic low voltage level (VL) to a logic high voltage level (VH) first, such that the transistor 832 and the transistor 850 are jointly activated to couple the global bit line GBL0 to the BBL 824 and further to the TBL 864. After the write enable signal WEN0 and the write enable signal WEN1 transition to VH, the BBL 824 and the TBL 864 transition from VL to VH according to the data value driven onto the global bit line GBL0. The read enable signal REN is maintained at VL throughout the write operation. After the write operation directed to the top memory cell 743 is completed, both the write enable signal WEN0 and the write enable signal WEN1 transition from VH back to VL.

Following the write operation directed to the top memory cell 743, the bottom memory cell 733 is written. Accordingly, the write enable signal WEN1 transitions from VL to VH first, such that the transistor 832 is activated to couple the global bit line GBL0 to the BBL 824, while the write enable signal WEN0 is maintained at VL to keep the transistor 850 deactivated. After the write enable signal WEN1 transitions to VH, the BBL 824 transitions from VL to VH according to the next data value driven onto the global bit line GBL0. The read enable signal REN is maintained at VL throughout the write operation. After the write operation directed to the bottom memory cell 733 is completed, the write enable signal WEN1 transitions from VH back to VL. In some embodiments, the voltage level developed on the BBL 824 is determined by the data value presented on the global bit line GBL0. For example, when writing a logic zero to the bottom memory cell 733, the global bit line GBL0 and the BBL 824 represent VL during the interval in which the write enable signal WEN1 is at VH, and when writing a logic one to the bottom memory cell 733, the global bit line GBL0 and the BBL 824 represent VH during the interval in which the write enable signal WEN1 is at VH.

Referring to FIG. 11, the memory layer 720H is shown as a representative example, with a first signal path, shown as fly bit line path 1110, directed to a read operation on the bottom memory cell 733, and a second signal path, shown as fly read bit line routing 1120, directed to a read operation on the top memory cell 743, in accordance with some embodiments. The TRBL 842 routes a far-side read path from the top array 740 across the bottom array 730 as one of the fly bit lines FBLs formed in a higher metallization layer, while the BRBL 802, routing a near-side read path from the bottom array 730 to the global bit line GBL0, is formed in a lower metallization layer.

During a read operation directed to the bottom memory cell 733, a data signal stored by the bottom memory cell 733 selected by assertion of the BWL0 826 propagates along the fly bit line path 1110 onto the BBL 824 (as a voltage signal), through the transistor 812 activated by the read enable signal REN, and onto the global bit line GBL0 as a current signal generated by the bottom gain cell 735 on the BRBL 802. Stated another way, the bottom gain cell 735 is configured to convert a voltage signal into a current signal. In some embodiments, the current level developed on the BRBL 802 is determined by the data value stored by the selected bottom memory cell 733. For example, when reading a logic low from the bottom memory cell 733, the BRBL 802 and the global bit line GBL0 represent a low current level (IL), and when reading a logic high from the bottom memory cell 733, the BRBL 802 and the global bit line GBL0 represent a high current level (IH).

During a read operation directed to the top memory cell 743, a data signal stored by the top memory cell 743 selected by assertion of the TWL0 866 propagates along the fly read bit line routing 1120 onto the TBL 864, through the transistor 852 activated by the read enable signal REN, and onto the global bit line GBL1 as a current signal generated by the top gain cell 745 on the TRBL 842. Stated another way, the top gain cell 745 is configured to convert a voltage signal into a current signal. In some embodiments, the current level developed on the TRBL 842 is determined by the data value stored by the selected top memory cell 743. For example, when reading a logic low from the top memory cell 743, the TRBL 842 and the global bit line GBL1 represent a low current level (IL), and when reading a logic high from the top memory cell 743, the TRBL 842 and the global bit line GBL1 represent a high current level (IH). In some embodiments, the TRBL 842 and the BRBL 802 are electrically decoupled from each other, such that a first data bit may be read from the bottom memory cell 733 along the fly bit line path 1110 and a second data bit may be read from the top memory cell 743 along the fly read bit line routing 1120 in parallel.

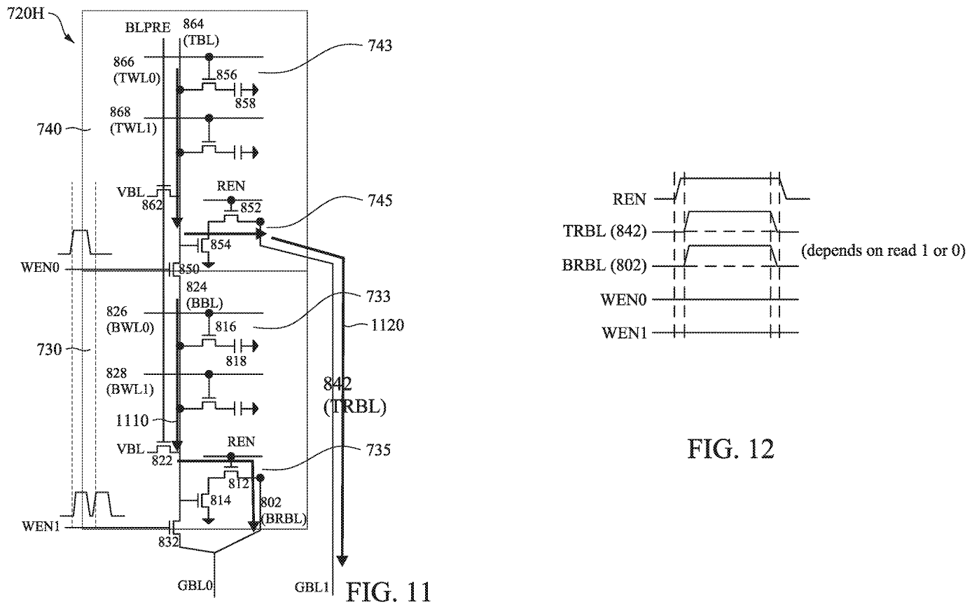

Referring to FIG. 12, a diagram illustrating waveforms of signals, while reading the bottom array 730 and the top array 740 in parallel through the BRBL 802 and the TRBL 842, is shown, in accordance with some embodiments. For example, the read enable signal REN, the signals present on the TRBL 842, the signals present on the BRBL 802, the write enable signal WEN0, and the write enable signal WEN1 are shown. It should be understood that the waveforms of FIG. 12 are provided merely for illustrative purposes, and do not intend to limit the scope of the present disclosure.

During a read operation, the read enable signal REN transitions from a logic low voltage level (VL) to a logic high voltage level (VH) first, such that the transistor 852 and the transistor 812 are activated to couple the TRBL 842 to the global bit line GBL1 and to couple the BRBL 802 to the global bit line GBL0, respectively. After the read enable signal REN transitions to VH, the TRBL 842 and the BRBL 802 transition from IL to IH according to respective data values stored by the selected top memory cell 743 and the selected bottom memory cell 733. The write enable signal WEN0 and the write enable signal WEN1 are maintained at VL throughout the read operation, such that the transistor 850 and the transistor 832 remain deactivated, and the write bit line paths are decoupled from the read current paths through the top gain cell 745 and the bottom gain cell 735. After the read operation is completed, the read enable signal REN transitions from VH back to VL. In some embodiments, the data values developed onto the TRBL 842 and the BRBL 802 are independent of each other, such that a first bit may be read from the top memory cell 743 along the fly read bit line routing 1120 and a second bit may be read concurrently from the bottom memory cell 733 along the fly bit line path 1110. For example, when reading a logic zero from the top memory cell 743 and a logic one from the bottom memory cell 733 in parallel, the TRBL 842 and the global bit line GBL1 represent IL while the BRBL 802 and the global bit line GBL0 represent IH during the interval in which the read enable signal REN is at VH.

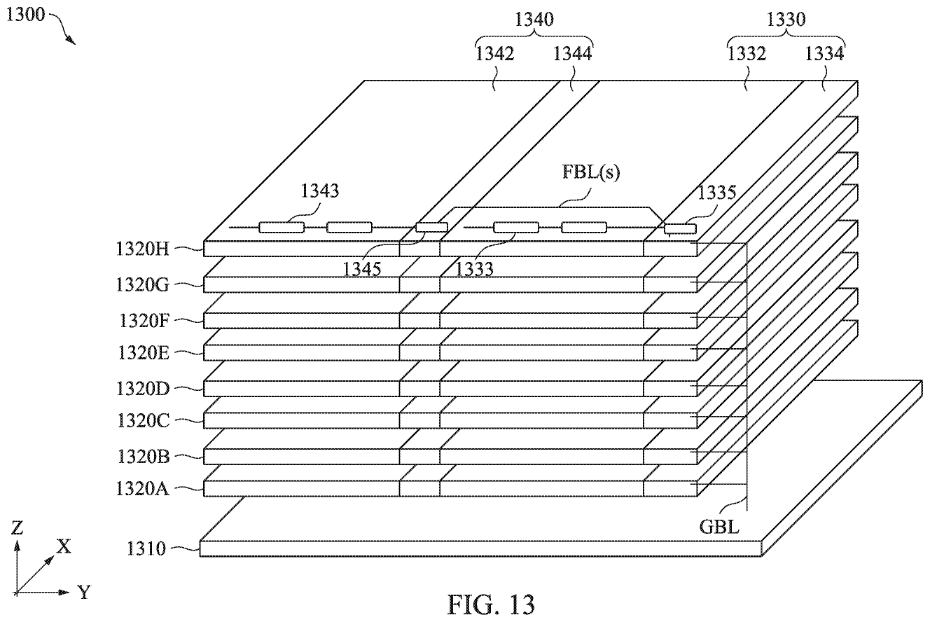

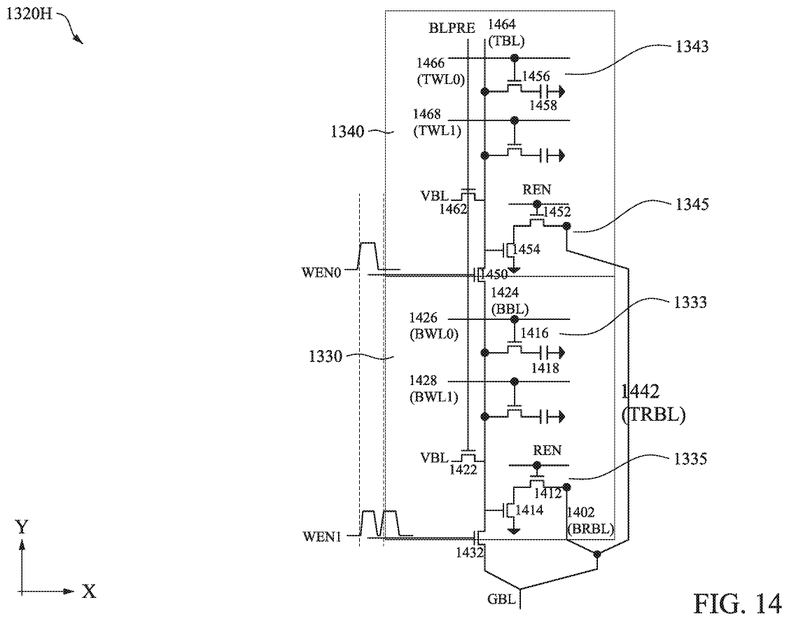

Referring to FIG. 13, a memory device 1300 can include a base die 1310 and a plurality of memory layers such as, for example, memory layer 1320A, memory layer 1320B, memory layer 1320C, memory layer 1320D, memory layer 1320E, memory layer 1320F, memory layer 1320G, and memory layer 1320H stacked over the base die 1310 along a vertical direction. The base die 1310 can include control, driver, and input/output (I/O) circuitry that facilitates read and write operations on the memory layer 1320A through memory layer 1320H. In some implementations, the memory layer 1320A through memory layer 1320H can be respectively formed in a plurality of metallization layers disposed over a major surface of the base die 1310, such that the memory layer 1320A through memory layer 1320H share the peripheral circuitry of the base die 1310. For example, the memory device 1300 can be arranged as an 8-stack memory, with each of the memory layer 1320A through memory layer 1320H providing a separate memory plane along the Z direction above the base die 1310 over the X-Y plane.

Each of the memory layers 1320A through memory layer 1320H can include a first array, shown as top array 1340, and a second array, shown as bottom array 1330, disposed adjacent to each other along a lateral direction parallel to the lengthwise direction of local bit lines. The top array 1340 can include a top memory cell region 1342 and a top gain cell region 1344, and the bottom array 1330 can include a bottom memory cell region 1332 and a bottom gain cell region 1334. In some implementations, the bottom memory cell region 1332 can include a plurality of bottom memory cells 1333 each operable to store a data bit, and the bottom gain cell region 1334 can include a plurality of bottom gain cells 1335 each operable to output a current representative of a stored logic state of a corresponding one of the bottom memory cells 1333. For example, the top memory cell region 1342 can include a plurality of top memory cells 1343 arranged along the lateral direction, and the top gain cell region 1344 can include a plurality of top gain cells 1345 that receive a voltage signal from the top memory cells 1343 and output a corresponding current signal. The top array 1340 and the bottom array 1330 can be disposed such that the top gain cell region 1344 is interposed between the top memory cell region 1342 and the bottom gain cell region 1334 along the lateral direction.

The memory device 1300 can include a global bit line GBL that vertically extends across the metallization layers in which the memory layer 1320A through memory layer 1320H are formed. The global bit line GBL can route read and write signals between the memory layer 1320A through memory layer 1320H and the base die 1310. In some implementations, the global bit line GBL can be disposed on a side of the memory layer 1320A through memory layer 1320H along the lateral direction, such that the bottom array 1330 is closer to the global bit line GBL than the top array 1340. For example, the bottom array 1330 can be a near-side array with respect to the global bit line GBL, and the top array 1340 can be a far-side array with respect to the global bit line GBL. In some embodiments, the global bit line GBL may be configured to route a merged current signal, formed by summing currents provided by the top gain cell 1345 of the top array 1340 and the bottom gain cell 1335 of the bottom array 1330, to the base die 1310 as a multi-level-cell current representative of a two-bit combination.

Each of the memory layer 1320A through memory layer 1320H can include one or more fly bit lines FBL(s) that route a far-side bit line path from the top array 1340 across the bottom array 1330 to the global bit line GBL. The fly bit lines FBL(s) can be formed in a higher metallization layer than the local bit lines that couple the top memory cells 1343 and the bottom memory cells 1333 along the lateral direction, such that the parasitic resistance and parasitic capacitance presented on the local bit lines are reduced. In some implementations, the FBL(s) can extend laterally over the bottom memory cell region 1332 and bottom gain cell region 1334 within a corresponding one of the memory layer 1320A through memory layer 1320H, and can couple to the global bit line GBL that may be formed as a vertical via disposed on the side of the bottom gain cell region 1334. In the illustrative example of FIG. 13, the FBL(s) can include a top read bit line placed on a metallization layer higher than another metallization layer, where other bit lines and/or word lines are formed, to facilitate a far-side read operation for the top array 1340, while a shared write bit line supports a sequential write function selectable between the top array 1340 and the bottom array 1330 under control of write enable signals generated by the base die 1310.

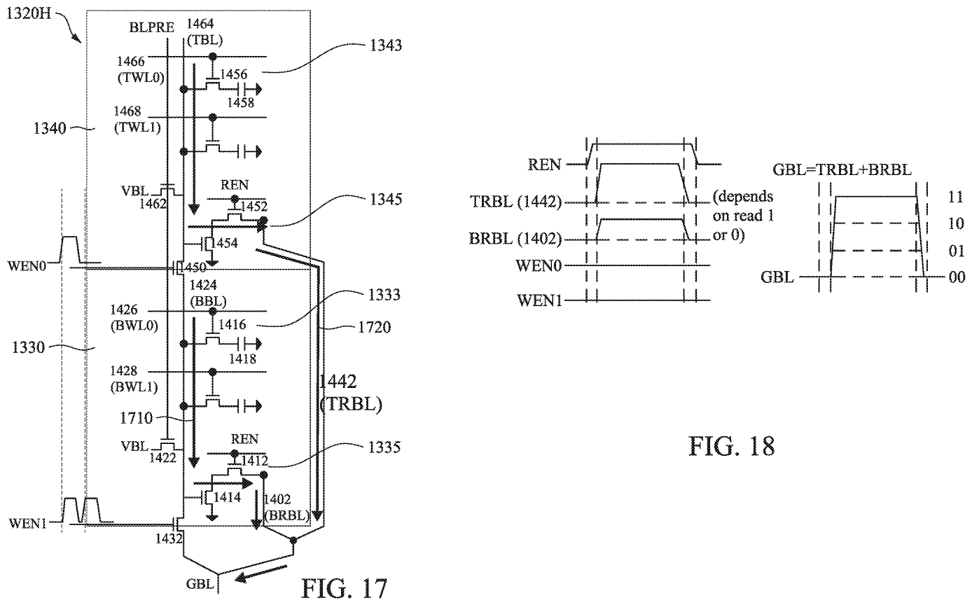

In some embodiments, the top gain cells 1345 of the top array 1340 may be sized larger than the bottom gain cells 1335 of the bottom array 1330, such that a current contribution provided by the top array 1340 onto the global bit line GBL is multiplied relative to a current contribution provided by the bottom array 1330. For example, a size parameter M may be equal to 2 for the top gain cells 1345 and equal to 1 for the bottom gain cells 1335, such that an output current of the top gain cells 1345 is doubled. Accordingly, the merged current on the global bit line GBL represents four current levels corresponding to stored two-bit combinations 00, 01, 10, and 11, which will be discussed further in FIGS. 17-18.

Referring to FIG. 14, a circuit diagram of the memory layer 1320H is shown as a representative example of the memory layers described with respect to FIG. 13, in accordance with some embodiments. The memory layer 1320H can include the top array 1340 and the bottom array 1330 disposed adjacent to each other along a lateral direction (e.g., the Y direction), with the top array 1340 being a far-side array relative to the global bit line GBL, and the bottom array 1330 being a near-side array relative to the global bit line GBL. The top array 1340 can include the top memory cell 1343 and the top gain cell 1345, and the bottom array 1330 can include the bottom memory cell 1333 and the bottom gain cell 1335. The memory layer 1320H can be coupled to the base die through the global bit line GBL, with the top read bit line 1442 routing a far-side read path from the top array 1340 across the bottom array 1330 as one of the fly bit lines FBLs formed on a higher metallization layer, while a shared write path supports a sequential write function selectable between the top array 1340 and the bottom array 1330 under control of write enable signals WEN0 and WEN1 generated by the base die.

Referring first to the bottom array 1330, the bottom gain cell 1335 includes transistor 1412 and transistor 1414 (e.g., both in n-type) coupled to each other in series. The transistor 1412 has a first source/drain terminal coupled to a local bit line 1402, sometimes referred to as bottom read bit line (BRBL) 1402, and a second source/drain terminal coupled to a first source/drain terminal of the transistor 1414. The BRBL 1402 is coupled to the global bit line GBL. The transistor 1414 has a second source/drain terminal coupled to ground. Further, the transistor 1412 has a gate terminal configured to receive a read enable signal REN, and the transistor 1414 has a gate terminal coupled to a local bit line 1424, sometimes referred to as bottom bit line (BBL) 1424. The BBL 1424 and the TBL 1464 may be formed as different portion of one local bit line, in some embodiments. In some embodiments, the BBL 1424 and a bit line voltage (VBL) can be coupled to first and second source/drain terminals of a transistor 1422, respectively, and the transistor 1422 has a gate terminal operating according to a bit line pre-charge signal BLPRE. In some embodiments, the BBL 1424 is coupled to the global bit line GBL through a transistor 1432, and the transistor 1432 has a gate terminal operating according to the write enable signal WEN1. Referring still to the bottom array 1330, the bottom memory cell 1333 includes a transistor 1416 and a capacitor 1418 coupled to each other in series. The transistor 1416 has its first and second source/drain terminals coupled to the BBL 1424 and one of the terminals of the capacitor 1418, respectively. The other terminal of the capacitor 1418 is coupled to ground. Further, a gate terminal of the transistor 1416 is coupled to a local word line 1426, sometimes referred to as bottom word line 0 (BWL0) 1426. By asserting the BWL0 1426, the transistor 1416 can be activated, which allows access to the bottom memory cell 1333.

Referring next to the top array 1340, the top gain cell 1345 includes transistors 1452 and 1454 (e.g., both in n-type) coupled to each other in series. The transistor 1452 has a first source/drain terminal coupled to a local bit line 1442, sometimes referred to as top read bit line (TRBL) 1442, and a second source/drain terminal coupled to a first source/drain terminal of the transistor 1454. The TRBL 1442, formed as one of the fly bit lines FBLs, is also coupled to the global bit line GBL, such that a current signal provided by the top gain cell 1345 is merged with a current signal provided by the bottom gain cell 1335 on the global bit line GBL. The transistor 1454 has a second source/drain terminal coupled to ground. Further, the transistor 1452 has a gate terminal configured to receive the read enable signal REN, and the transistor 1454 has a gate terminal coupled to a local bit line 1464, sometimes referred to as top bit line (TBL) 1464. In some embodiments, the TBL 1464 and the bit line voltage (VBL) can be coupled to first and second source/drain terminals of a transistor 1462, respectively, and the transistor 1462 has a gate terminal operating according to the bit line pre-charge signal BLPRE. In some embodiments, the top gain cell 1345 is sized larger than the bottom gain cell 1335, such that the current signal provided by the top gain cell 1345 onto the global bit line GBL is multiplied relative to the current signal provided by the bottom gain cell 1335. For example, a size parameter M may be equal to 2 for the top gain cell 1345 and equal to 1 for the bottom gain cell 1335, such that an output current of the top gain cell 1345 is doubled relative to an output current of the bottom gain cell 1335.

In some embodiments, the TBL 1464 is coupled to the BBL 1424 through a transistor 1450, and the transistor 1450 has a gate terminal operating according to the write enable signal WEN0. In some embodiments, the transistor 1450 and the transistor 1432 are jointly activated to propagate a data signal from the global bit line GBL to the TBL 1464 during a top bit line write operation, while the transistor 1432 alone is activated during a bottom bit line write operation, such that a single write bit line path supports a sequential write function selectable between the top array 1340 and the bottom array 1330. Referring still to the top array 1340, the top memory cell 1343 includes a transistor 1456 and a capacitor 1458 coupled to each other in series. The transistor 1456 has its first and second source/drain terminals coupled to the TBL 1464 and one of the terminals of the capacitor 1458, respectively. The other terminal of the capacitor 1458 is coupled to ground. Further, a gate terminal of the transistor 1456 is coupled to a local word line 1466, sometimes referred to as top word line 0 (TWL0) 1466. By asserting the TWL0 1466, the transistor 1456 can be activated, which allows access to the top memory cell 1343. In some embodiments, the memory layer 1320H further includes a local word line 1468, sometimes referred to as top word line 1 (TWL1) 1468, coupled to a gate terminal of another transistor of the top memory cell region 1342, and a local word line 1428, sometimes referred to as bottom word line 1 (BWL1) 1428, coupled to a gate terminal of another transistor of the bottom memory cell region 1332.

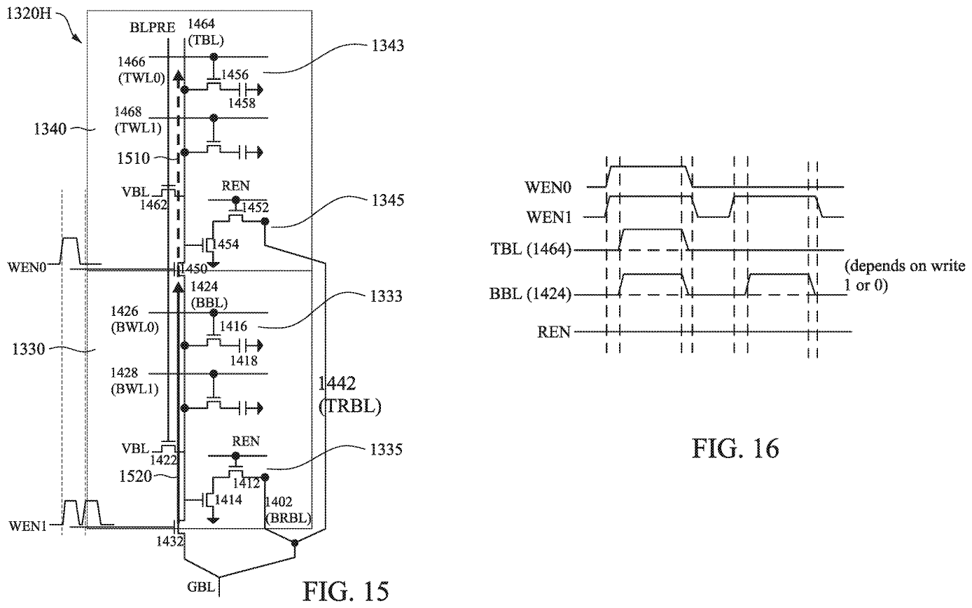

Referring to FIG. 15, the memory layer 1320H is shown as a representative example, with a first signal path, shown as signal path 1510, directed to a write operation on the top memory cell 1343, and a second signal path, shown as signal path 1520, directed to a write operation on the bottom memory cell 1333, in accordance with some embodiments. In the example of FIG. 15, the write operation performed on the bottom memory cell 1333 may be subsequent to the write operation performed on the top memory cell 1343. As such, the signal path 1510 and the signal path 1520 may share the global bit line GBL and the BBL 1424 as a common write bit line path, such that a sequential write function is selectable between the top array 1340 and the bottom array 1330 under control of the write enable signal WEN0 and the write enable signal WEN1 generated by the base die.

During a write operation directed to the top memory cell 1343, a data signal provided on the global bit line GBL propagates along the signal path 1510 through the transistor 1432 activated by the write enable signal WEN1, onto the BBL 1424, through the transistor 1450 activated by the write enable signal WEN0, and further onto the TBL 1464, and into the top memory cell 1343 selected by assertion of the TWL0 1466. In some embodiments, the voltage level developed on the BBL 1424 and the TBL 1464 is determined by the data value presented on the global bit line GBL. For example, when writing a logic high to the top memory cell 1343, the global bit line GBL, the BBL 1424, and the TBL 1464 represent a logic high voltage level, such that the capacitor 1458 of the selected top memory cell 1343 is charged through the transistor 1456 activated by the corresponding TWL0 1466.

During a subsequent write operation directed to the bottom memory cell 1333, a next data signal provided on the global bit line GBL propagates along the signal path 1520 through the transistor 1432 activated by the write enable signal WEN1, onto the BBL 1424, and into the bottom memory cell 1333 selected by assertion of the BWL0 1426. In some embodiments, the transistor 1450 may remain deactivated during the write operation directed to the bottom memory cell 1333, such that the TBL 1464 is decoupled from the BBL 1424 and the top memory cell 1343 is not disturbed by the data signal propagating along the signal path 1520. For example, when writing a logic low to the bottom memory cell 1333, the global bit line GBL and the BBL 1424 represent a logic low voltage level, such that the capacitor 1418 of the selected bottom memory cell 1333 is discharged through the transistor 1416 activated by the corresponding BWL0 1426. In some embodiments, the signal path 1510 and the signal path 1520 are executed sequentially, such that the top memory cell 1343 is written first and the bottom memory cell 1333 is written thereafter using the single write bit line path.

Referring to FIG. 16, a diagram illustrating waveforms of signals, while sequentially writing the top array 1340 and then the bottom array 1330 through the shared write bit line path, is shown, in accordance with some embodiments. For example, the write enable signal WEN0, the write enable signal WEN1, the signals present on the TBL 1464, the signals present on the BBL 1424, and the read enable signal REN are shown. It should be understood that the waveforms of FIG. 16 are provided merely for illustrative purposes, and do not intend to limit the scope of the present disclosure.

During a sequential write operation, the top memory cell 1343 may be written first. Accordingly, both the write enable signal WEN0 and the write enable signal WEN1 transition from a logic low voltage level (VL) to a logic high voltage level (VH) first, such that the transistor 1432 and the transistor 1450 are jointly activated to couple the global bit line GBL to the BBL 1424 and further to the TBL 1464. After the write enable signal WEN0 and the write enable signal WEN1 transition to VH, the BBL 1424 and the TBL 1464 transition from VL to VH according to the data value driven onto the global bit line GBL. The read enable signal REN is maintained at VL throughout the write operation. After the write operation directed to the top memory cell 1343 is completed, both the write enable signal WEN0 and the write enable signal WEN1 transition from VH back to VL.

Following the write operation directed to the top memory cell 1343, the bottom memory cell 1333 is written. Accordingly, the write enable signal WEN1 transitions from VL to VH first, such that the transistor 1432 is activated to couple the global bit line GBL to the BBL 1424, while the write enable signal WEN0 is maintained at VL to keep the transistor 1450 deactivated. After the write enable signal WEN1 transitions to VH, the BBL 1424 transitions from VL to VH according to the next data value driven onto the global bit line GBL. The read enable signal REN is maintained at VL throughout the write operation. After the write operation directed to the bottom memory cell 1333 is completed, the write enable signal WEN1 transitions from VH back to VL. In some embodiments, the voltage level developed on the BBL 1424 is determined by the data value presented on the global bit line GBL. For example, when writing a logic zero to the bottom memory cell 1333, the global bit line GBL and the BBL 1424 represent VL during the interval in which the write enable signal WEN1 is at VH, and when writing a logic one to the bottom memory cell 1333, the global bit line GBL and the BBL 1424 represent VH during the interval in which the write enable signal WEN1 is at VH.

Referring to FIG. 16, a diagram illustrating waveforms of signals, while sequentially writing the top array 1340 and then the bottom array 1330 through the shared write bit line path, is shown, in accordance with some embodiments. For example, the write enable signal WEN0, the write enable signal WEN1, the signals present on the TBL 1464, the signals present on the BBL 1424, and the read enable signal REN are shown. It should be understood that the waveforms of FIG. 16 are provided merely for illustrative purposes, and do not intend to limit the scope of the present disclosure.

During a sequential write operation, the top memory cell 1343 may be written first. Accordingly, both the write enable signal WEN0 and the write enable signal WEN1 transition from a logic low voltage level (VL) to a logic high voltage level (VH) first, such that the transistor 1432 and the transistor 1450 are jointly activated to couple the global bit line GBL to the BBL 1424 and further to the TBL 1464. After the write enable signal WEN0 and the write enable signal WEN1 transition to VH, the BBL 1424 and the TBL 1464 transition from VL to VH according to the data value driven onto the global bit line GBL. The read enable signal REN is maintained at VL throughout the write operation. After the write operation directed to the top memory cell 1343 is completed, both the write enable signal WEN0 and the write enable signal WEN1 transition from VH back to VL.

Following the write operation directed to the top memory cell 1343, the bottom memory cell 1333 is written. Accordingly, the write enable signal WEN1 transitions from VL to VH first, such that the transistor 1432 is activated to couple the global bit line GBL to the BBL 1424, while the write enable signal WEN0 is maintained at VL to keep the transistor 1450 deactivated. After the write enable signal WEN1 transitions to VH, the BBL 1424 transitions from VL to VH according to the next data value driven onto the global bit line GBL. The read enable signal REN is maintained at VL throughout the write operation. After the write operation directed to the bottom memory cell 1333 is completed, the write enable signal WEN1 transitions from VH back to VL. In some embodiments, the voltage level developed on the BBL 1424 is determined by the data value presented on the global bit line GBL. For example, when writing a logic zero to the bottom memory cell 1333, the global bit line GBL and the BBL 1424 represent VL during the interval in which the write enable signal WEN1 is at VH, and when writing a logic one to the bottom memory cell 1333, the global bit line GBL and the BBL 1424 represent VH during the interval in which the write enable signal WEN1 is at VH.

Referring to FIG. 17, the memory layer 1320H is shown as a representative example, with a first signal path, shown as bottom local read current path 1710, directed to a read operation on the bottom memory cell 1333, and a second signal path, shown as fly read bit line path 1720, directed to a read operation on the top memory cell 1343, in accordance with some embodiments. The TRBL 1442 routes a far-side read path from the top array 1340 across the bottom array 1330 as one of the fly bit lines FBLs formed in a higher metallization layer, while the BRBL 1402, routing a near-side read path from the bottom array 1330 to the global bit line GBL, is formed in a lower metallization layer. The TRBL 1442 and the BRBL 1402 are both coupled to the global bit line GBL, such that a current signal provided by the top gain cell 1345 is merged with a current signal provided by the bottom gain cell 1335 on the global bit line GBL to form a multi-level-cell current.

During a read operation directed to the bottom memory cell 1333, a data signal stored by the bottom memory cell 1333 selected by assertion of the BWL0 1426 propagates along the bottom local read current path 1710 onto the BBL 1424 (as a voltage signal), through the transistor 1412 activated by the read enable signal REN, and onto the global bit line GBL as a current signal generated by the bottom gain cell 1335 on the BRBL 1402. Stated another way, the bottom gain cell 1335 is configured to convert a voltage signal into a current signal. In some embodiments, the current level developed on the BRBL 1402 is determined by the data value stored by the selected bottom memory cell 1333. For example, when reading a logic low from the bottom memory cell 1333, the BRBL 1402 represents a low current level (IL), and when reading a logic high from the bottom memory cell 1333, the BRBL 1402 represents a high current level (IH).

During a read operation directed to the top memory cell 1343, a data signal stored by the top memory cell 1343 selected by assertion of the TWL0 1466 propagates along the fly read bit line path 1720 onto the TBL 1464, through the transistor 1452 activated by the read enable signal REN, and onto the global bit line GBL as a current signal generated by the top gain cell 1345 on the TRBL 1442. Stated another way, the top gain cell 1345 is configured to convert a voltage signal into a current signal. In some embodiments, the top gain cell 1345 is sized larger than the bottom gain cell 1335, such that the current level developed on the TRBL 1442 is multiplied relative to the current level developed on the BRBL 1402. For example, when the size parameter M is equal to 2 for the top gain cell 1345 and equal to 1 for the bottom gain cell 1335, the TRBL 1442 represents a gained low current level when reading a logic low from the top memory cell 1343, and the TRBL 1442 represents a doubled high current level (2×IH) when reading a logic high from the top memory cell 1343. In some embodiments, the current signal on the TRBL 1442 and the current signal on the BRBL 1402 are summed on the global bit line GBL, such that the global bit line GBL represents a multi-level-cell current equal to the sum of the current on the TRBL 1442 and the current on the BRBL 1402.

Referring to FIG. 18, a diagram illustrating waveforms of signals, while reading the bottom array 1330 and the top array 1340 through the BRBL 1402 and the TRBL 1442 to generate the multi-level-cell current on the global bit line GBL, is shown, in accordance with some embodiments. For example, the read enable signal REN, the signals present on the TRBL 1442, the signals present on the BRBL 1402, the signals present on the global bit line GBL, the write enable signal WEN0, and the write enable signal WEN1 are shown. It should be understood that the waveforms of FIG. 18 are provided merely for illustrative purposes, and do not intend to limit the scope of the present disclosure.

During a read operation, the read enable signal REN transitions from a logic low voltage level (VL) to a logic high voltage level (VH) first, such that the transistor 1452 and the transistor 1412 are activated to couple the TRBL 1442 and the BRBL 1402 to the global bit line GBL. After the read enable signal REN transitions to VH, the BRBL 1402 transitions from IL to IH according to the data value stored by the selected bottom memory cell 1333, and the TRBL 1442 transitions from IL to 2×IH according to the data value stored by the selected top memory cell 1343 due to the size parameter M being equal to 2 for the top gain cell 1345. The write enable signal WEN0 and the write enable signal WEN1 are maintained at VL throughout the read operation, such that the transistor 1450 and the transistor 1432 remain deactivated, and the write bit line paths are decoupled from the read current paths through the top gain cell 1345 and the bottom gain cell 1335. After the read operation is completed, the read enable signal REN transitions from VH back to VL. In some embodiments, the current level developed on the global bit line GBL is the sum of the current level on the TRBL 1442 and the current level on the BRBL 1402, such that the global bit line GBL may represent four current levels corresponding to stored two-bit combinations. For example, when a logic zero is read from the top memory cell 1343 and a logic zero is read from the bottom memory cell 1333, the TRBL 1442 represents a gained logic low and the BRBL 1402 represents IL, such that the global bit line GBL is at IL to represent bit 00. For example, when a logic zero is read from the top memory cell 1343 and a logic one is read from the bottom memory cell 1333, the TRBL 1442 represents a gained logic low and the BRBL 1402 represents IH, such that the global bit line GBL is at IH to represent bit 01. For example, when a logic one is read from the top memory cell 1343 and a logic zero is read from the bottom memory cell 1333, the TRBL 1442 represents 2×IH and the BRBL 1402 represents IL, such that the global bit line GBL is at 2×IH to represent bit 10. For example, when a logic one is read from the top memory cell 1343 and a logic one is read from the bottom memory cell 1333, the TRBL 1442 represents 2×IH and the BRBL 1402 represents IH, such that the global bit line GBL is at 3×IH to represent bit 11. In some embodiments, the base die 1310 detects the four current levels on the global bit line GBL to distinguish the two-bit combinations 00, 01, 10, and 11.

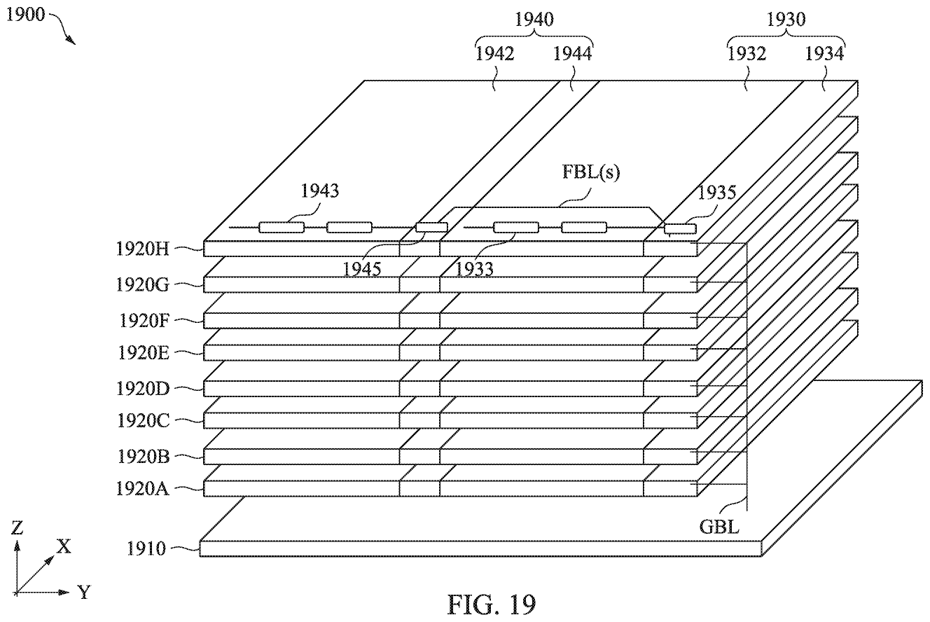

Referring to FIG. 19, a memory device 1900 can include a base die 1910 and a plurality of memory layers such as, for example, memory layer 1920A, memory layer 1920B, memory layer 1920C, memory layer 1920D, memory layer 1920E, memory layer 1920F, memory layer 1920G, and memory layer 1920H stacked over the base die 1910 along a vertical direction. The base die 1910 can include control, driver, and input/output (I/O) circuitry that facilitates read and write operations on the memory layer 1920A through memory layer 1920H. In some implementations, the memory layer 1920A through memory layer 1920H can be respectively formed in a plurality of metallization layers disposed over a major surface of the base die 1910, such that the memory layer 1920A through memory layer 1920H share the peripheral circuitry of the base die 1910. For example, the memory device 1900 can be arranged as an 8-stack memory, with each of the memory layer 1920A through memory layer 1920H providing a separate memory plane along the Z direction above the base die 1910 over the X-Y plane.

Each of the memory layers 1920A through memory layer 1920H can include a first memory-array portion, shown as top array 1940, and a second memory-array portion, shown as bottom array 1930, disposed adjacent to each other along a lateral direction parallel to the lengthwise direction of local bit lines. The top array 1940 can include a top memory cell region 1942 and a top gain cell region 1944, and the bottom array 1930 can include a bottom memory cell region 1932 and a bottom gain cell region 1934. In some implementations, the bottom memory cell region 1932 can include a plurality of memory cells of a bottom memory cell 1933 each operable to store a data bit, and the bottom gain cell region 1934 can include a plurality of gain cells of a bottom gain cell 1935 each operable to output a current representative of a stored logic state of a corresponding memory cell of the bottom memory cell 1933.

For example, the top memory cell region 1942 can include a plurality of memory cells 1943 arranged along the lateral direction, and the top gain cell region 1944 can include a plurality of gain cells 1945 that receive a voltage signal from the memory cells 1943 and output a corresponding current signal. The top array 1940 and the bottom array 1930 can be disposed such that the top gain cell region 1944 is interposed between the top memory cell region 1942 and the bottom gain cell region 1934 along the lateral direction. The memory device 1900 can include a global bit line GBL that vertically extends across the metallization layers in which the memory layer 1920A through memory layer 1920H are formed. The global bit line GBL can route read and write signals between the memory layer 1920A through memory layer 1920H and the base die 1910.

In some implementations, the global bit line GBL can be disposed on a side of the memory layer 1920A through memory layer 1920H along the lateral direction, such that the bottom array 1930 is closer to the global bit line GBL than the top array 1940. For example, the bottom array 1930 can be a near-side array with respect to the global bit line GBL, and the top array 1940 can be a far-side array with respect to the global bit line GBL. In some embodiments, the global bit line GBL may be configured to route a single-bit current signal, provided selectively by the top gain cell 1945 of the top array 1940 or the bottom gain cell 1935 of the bottom array 1930, to the base die 1910 under selective word line control generated by the base die 1910.

Each of the memory layer 1920A through memory layer 1920H can include one or more fly bit lines FBL(s) that route a far-side bit line path from the top array 1940 across the bottom array 1930 to the global bit line GBL. The fly bit lines FBL(s) can be formed in a higher metallization layer than the local bit lines that couple the memory cells of the top memory cell 1943 and the memory cells of the bottom memory cell 1933 along the lateral direction, such that the parasitic resistance and parasitic capacitance presented on the local bit lines are reduced. In some implementations, the FBL(s) can extend laterally over the bottom memory cell region 1932 and the bottom gain cell region 1934 within a corresponding one of the memory layer 1920A through memory layer 1920H, and can couple to the global bit line GBL that may be formed as a vertical via disposed on the side of the bottom gain cell region 1934.

In the illustrative example of FIG. 19, the FBL(s) can include a shared read bit line placed on a metallization layer higher than another metallization layer, where other bit lines and/or word lines are formed, to facilitate a far-side read operation for the top array 1940, while a shared write bit line supports a one-bit write function selectable between the top array 1940 and the bottom array 1930 under control of write enable signals generated by the base die 1910. In some embodiments, the shared read bit line is reused for a one-bit read operation on the top array 1940 and a one-bit read operation on the bottom array 1930 under control of a top read word line signal and a bottom read word line signal generated by the base die 1910, such that the top gain cell 1945 propagates a current signal along the top gain cell 1945 during a top bit line read and the bottom gain cell 1935 propagates a current signal along the bottom gain cell 1935 during a bottom bit line read.

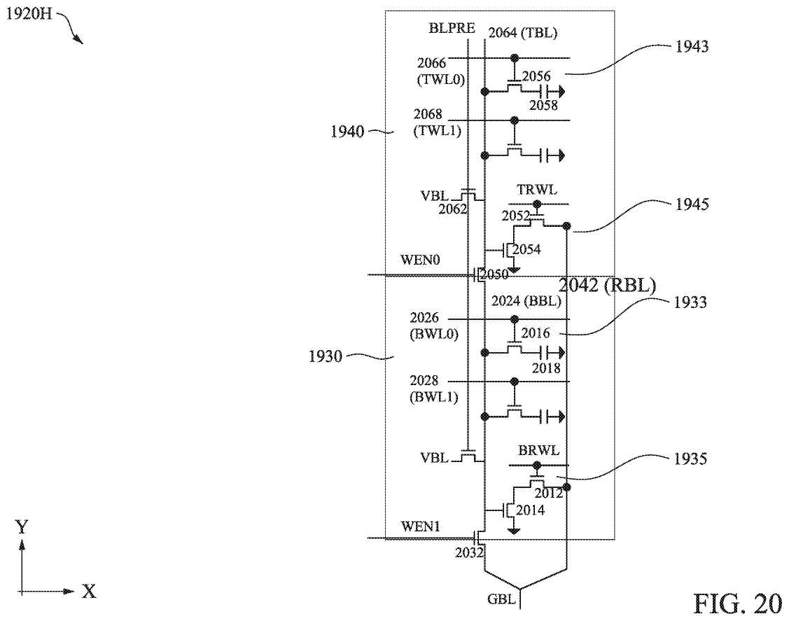

Referring to FIG. 20, a circuit diagram of the memory layer 1920H is shown as a representative example of the memory layers described with respect to FIG. 19, in accordance with some embodiments. The memory layer 1920H can include the top array 1940 and the bottom array 1930 disposed adjacent to each other along a lateral direction (e.g., Y direction), with the top array 1940 being a far-side array relative to the global bit line GBL, and the bottom array 1930 being a near-side array relative to the global bit line GBL. The top array 1940 can include the top memory cell 1943 and the top gain cell 1945, and the bottom array 1930 can include the bottom memory cell 1933 and the bottom gain cell 1935. The memory layer 1920H can be coupled to the base die through the global bit line GBL and a shared read bit line that routes a far-side read path from the top array 1940 across the bottom array 1930 as one of the fly bit lines FBLs formed on a higher metallization layer, while a shared write path supports a one-bit write function selectable between the top array 1940 and the bottom array 1930 under control of write enable signals WEN0 and WEN1 generated by the base die.

Referring first to the bottom array 1930, the bottom gain cell 1935 includes transistor 2012 and transistor 2014 (e.g., both in n-type) coupled to each other in series. The transistor 2012 has a first source/drain terminal coupled to a local bit line 2042, sometimes referred to as read bit line (RBL) 2042, and a second source/drain terminal coupled to a first source/drain terminal of the transistor 2014. The transistor 2014 has a second source/drain terminal coupled to ground. Further, the transistor 2012 has a gate terminal configured to receive a bottom read word line signal BRWL, and the transistor 2014 has a gate terminal coupled to a local bit line 2024, sometimes referred to as bottom bit line (BBL) 2024. The BBL 2024 and the TBL 2064 may be formed as different portion of one local bit line, in some embodiments. In some embodiments, the BBL 2024 and a bit line voltage (VBL) can be coupled to first and second source/drain terminals of a transistor, respectively, the transistor having a gate terminal operating according to a bit line pre-charge signal BLPRE.

In some embodiments, the BBL 2024 is coupled to the global bit line GBL through a transistor 2032, and the transistor 2032 has a gate terminal operating according to the write enable signal WEN1. Referring still to the bottom array 1930, the bottom memory cell 1933 includes a transistor 2016 and a capacitor 2018 coupled to each other in series. The transistor 2016 has its first and second source/drain terminals coupled to the BBL 2024 and one of the terminals of the capacitor 2018, respectively. The other terminal of the capacitor 2018 is coupled to ground. Further, a gate terminal of the transistor 2016 is coupled to a local word line 2026, sometimes referred to as bottom word line 0 (BWL0) 2026. By asserting the BWL0 2026, the transistor 2016 can be activated, which allows access to the bottom memory cell 1933. In some embodiments, the memory layer 1920H further includes a local word line 2028, sometimes referred to as bottom word line 1 (BWL1) 2028, coupled to a gate terminal of another transistor of the bottom memory cell region 1932.

Referring next to the top array 1940, the top gain cell 1945 includes transistor 2052 and transistor 2054 (e.g., both in n-type) coupled to each other in series. The transistor 2052 has a first source/drain terminal coupled to the RBL 2042 and a second source/drain terminal coupled to a first source/drain terminal of the transistor 2054. In some embodiments, the RBL 2042, formed as one of the fly bit lines FBLs, is shared between the bottom array 1930 and the top array 1940, such that the RBL 2042 provides a common read current path to the global bit line GBL for a one-bit read operation selectable between the top array 1940 and the bottom array 1930. The transistor 2054 has a second source/drain terminal coupled to ground. Further, the transistor 2052 has a gate terminal configured to receive a top read word line signal TRWL, distinct from the bottom read word line signal BRWL received at the gate terminal of the transistor 2012, such that the transistor 2052 and the transistor 2012 are selectively activated by the base die to couple either the top gain cell 1945 or the bottom gain cell 1935 to the RBL 2042 during a read operation. The transistor 2054 has a gate terminal coupled to a local bit line 2064, sometimes referred to as top bit line (TBL) 2064. In some embodiments, the TBL 2064 and the bit line voltage (VBL) can be coupled to first and second source/drain terminals of a transistor, respectively, the transistor 2062 having a gate terminal operating according to the bit line pre-charge signal BLPRE.

In some embodiments, the TBL 2064 is coupled to the BBL 2024 through a transistor 2050, and the transistor 2050 has a gate terminal operating according to the write enable signal WEN0. In some embodiments, the transistor 2050 and the transistor 2032 are jointly activated to propagate a data signal from the global bit line GBL to the TBL 2064 during a top bit line write operation, while the transistor 2032 alone is activated during a bottom bit line write operation, such that a single write bit line path supports a one-bit write function selectable between the top array 1940 and the bottom array 1930. Referring still to the top array 1940, the top memory cell 1943 includes a transistor 2056 and a capacitor 2058 coupled to each other in series. The transistor 2056 has its first and second source/drain terminals coupled to the TBL 2064 and one of the terminals of the capacitor 2058, respectively. The other terminal of the capacitor 2058 is coupled to ground. Further, a gate terminal of the transistor 2056 is coupled to a local word line 2066, sometimes referred to as top word line 0 (TWL0) 2066. By asserting the TWL0 2066, the transistor 2056 can be activated, which allows access to the top memory cell 1943. In some embodiments, the memory layer 1920H further includes a local word line 2068, sometimes referred to as top word line 1 (TWL1) 2068, coupled to a gate terminal of another transistor of the top memory cell region 1942.

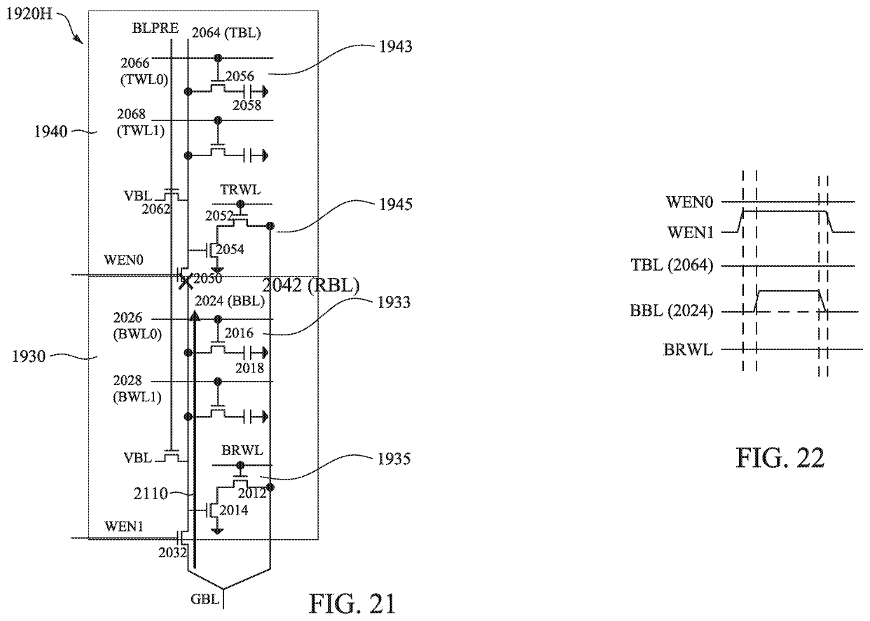

Referring to FIG. 21, the memory layer 1920H is shown as a representative example, with a signal path, shown as signal path 2110, directed to a write operation on the bottom memory cell 1933, in accordance with some embodiments. The signal path 2110 shares the global bit line GBL and the BBL 2024 as a write bit line path, such that a one-bit write function directed to the bottom array 1930 is performed under control of the write enable signal WEN0 and the write enable signal WEN1 generated by the base die.

During a write operation directed to the bottom memory cell 1933, a data signal provided on the global bit line GBL propagates along the signal path 2110 through the transistor 2032 activated by the write enable signal WEN1, onto the BBL 2024, and into the bottom memory cell 1933 selected by assertion of the BWL0 2026. In some embodiments, the transistor 2050 remains deactivated by the write enable signal WEN0 during the write operation directed to the bottom memory cell 1933, such that the TBL 2064 is decoupled from the BBL 2024 and the top memory cell 1943 is not disturbed by the data signal propagating along the signal path 2110. In some embodiments, the voltage level developed on the BBL 2024 is determined by the data value presented on the global bit line GBL. For example, when writing a logic low to the bottom memory cell 1933, the global bit line GBL and the BBL 2024 represent a logic low voltage level, such that the capacitor 2018 of the selected bottom memory cell 1933 is discharged through the transistor 2016 activated by the corresponding BWL0 2026. For example, when writing a logic high to the bottom memory cell 1933, the global bit line GBL and the BBL 2024 represent a logic high voltage level, such that the capacitor 2018 of the selected bottom memory cell 1933 is charged through the transistor 2016 activated by the corresponding BWL0 2026.

Referring to FIG. 22, a diagram illustrating waveforms of signals, while writing the bottom memory cell 1933 through the signal path 2110, is shown, in accordance with some embodiments. For example, the write enable signal WEN0, the write enable signal WEN1, the signals present on the TBL 2064, the signals present on the BBL 2024, and the bottom read word line signal BRWL are shown. It should be understood that the waveforms of FIG. 22 are provided merely for illustrative purposes, and do not intend to limit the scope of the present disclosure.

During a bottom bit line write operation, the write enable signal WEN1 transitions from a logic low voltage level (VL) to a logic high voltage level (VH) first, such that the transistor 2032 is activated to couple the global bit line GBL to the BBL 2024. The write enable signal WEN0 is maintained at VL throughout the bottom bit line write operation, such that the transistor 2050 remains deactivated and the TBL 2064 is decoupled from the BBL 2024. After the write enable signal WEN1 transitions to VH, the BBL 2024 transitions from VL to VH according to the data value driven onto the global bit line GBL. The bottom read word line signal BRWL is maintained at VL throughout the bottom bit line write operation, such that the transistor 2012 remains deactivated and the RBL 2042 is decoupled from the read current path through the bottom gain cell 1935. After the bottom bit line write operation is completed, the write enable signal WEN1 transitions from VH back to VL. In some embodiments, the voltage level developed on the BBL 2024 is determined by the data value presented on the global bit line GBL. For example, when writing a logic zero to the bottom memory cell 1933, the global bit line GBL and the BBL 2024 represent VL during the interval in which the write enable signal WEN1 is at VH. For example, when writing a logic one to the bottom memory cell 1933, the global bit line GBL and the BBL 2024 represent VH during the interval in which the write enable signal WEN1 is at VH.

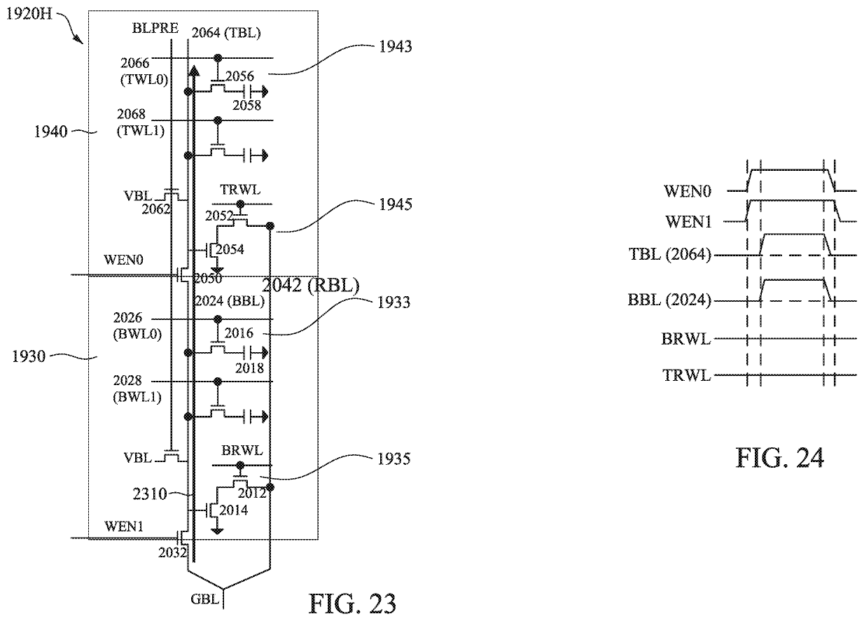

Referring to FIG. 23, the memory layer 1920H is shown as a representative example, with a signal path, shown as signal path 2310, directed to a write operation on the top memory cell 1943, in accordance with some embodiments. The signal path 2310 shares the global bit line GBL, the BBL 2024, and the TBL 2064 as a write bit line path, such that a one-bit write function directed to the top array 1940 is performed under control of the write enable signal WEN0 and the write enable signal WEN1 generated by the base die.

During a write operation directed to the top memory cell 1943, a data signal provided on the global bit line GBL propagates along the signal path 2310 through the transistor 2032 activated by the write enable signal WEN1, onto the BBL 2024, through the transistor 2050 activated by the write enable signal WEN0, and further onto the TBL 2064, and into the top memory cell 1943 selected by assertion of the TWL0 2066. In some embodiments, the voltage level developed on the BBL 2024 and the TBL 2064 is determined by the data value presented on the global bit line GBL. For example, when writing a logic low to the top memory cell 1943, the global bit line GBL, the BBL 2024, and the TBL 2064 represent a logic low voltage level, such that the capacitor 2058 of the selected top memory cell 1943 is discharged through the transistor 2056 activated by the corresponding TWL0 2066. For example, when writing a logic high to the top memory cell 1943, the global bit line GBL, the BBL 2024, and the TBL 2064 represent a logic high voltage level, such that the capacitor 2058 of the selected top memory cell 1943 is charged through the transistor 2056 activated by the corresponding TWL0 2066.

Referring to FIG. 24, a diagram illustrating waveforms of signals, while writing the top memory cell 1943 through the signal path 2310, is shown, in accordance with some embodiments. For example, the write enable signal WEN0, the write enable signal WEN1, the signals present on the TBL 2064, the signals present on the BBL 2024, the bottom read word line signal BRWL, and the top read word line signal TRWL are shown. It should be understood that the waveforms of FIG. 24 are provided merely for illustrative purposes, and do not intend to limit the scope of the present disclosure.

During a top bit line write operation, both the write enable signal WEN0 and the write enable signal WEN1 transition from a logic low voltage level (VL) to a logic high voltage level (VH) first, such that the transistor 2032 and the transistor 2050 are jointly activated to couple the global bit line GBL to the BBL 2024 and further to the TBL 2064. After the write enable signal WEN0 and the write enable signal WEN1 transition to VH, the BBL 2024 and the TBL 2064 transition from VL to VH according to the data value driven onto the global bit line GBL. The bottom read word line signal BRWL and the top read word line signal TRWL are maintained at VL throughout the top bit line write operation, such that the transistor 2012 and the transistor 2052 remain deactivated and the RBL 2042 is decoupled from the read current paths through the bottom gain cell 1935 and the top gain cell 1945. After the top bit line write operation is completed, both the write enable signal WEN0 and the write enable signal WEN1 transition from VH back to VL. In some embodiments, the voltage level developed on the BBL 2024 and the TBL 2064 is determined by the data value presented on the global bit line GBL. For example, when writing a logic zero to the top memory cell 1943, the global bit line GBL, the BBL 2024, and the TBL 2064 represent VL during the interval in which the write enable signal WEN0 and the write enable signal WEN1 are at VH. For example, when writing a logic one to the top memory cell 1943, the global bit line GBL, the BBL 2024, and the TBL 2064 represent VH during the interval in which the write enable signal WEN0 and the write enable signal WEN1 are at VH.

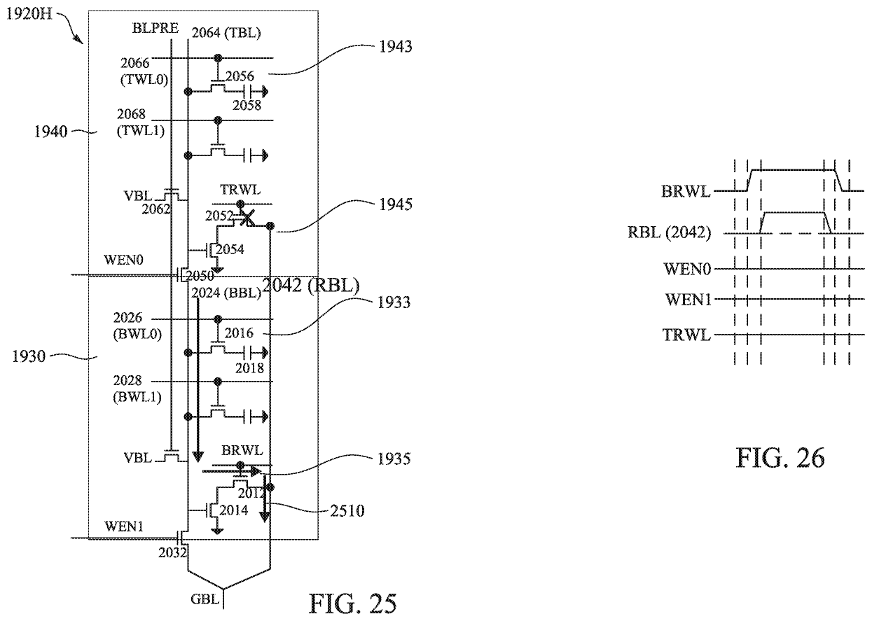

Referring to FIG. 25, the memory layer 1920H is shown as a representative example, with a signal path, shown as 2510, directed to a read operation on the bottom memory cell 1933, in accordance with some embodiments. The RBL 2042 routes a read current path from the bottom gain cell 1935 to the global bit line GBL as one of the fly bit lines FBLs formed in a higher metallization layer, and is shared between the bottom array 1930 and the top array 1940 such that a one-bit read function directed to the bottom array 1930 is performed under control of the bottom read word line signal BRWL generated by the base die.

During a read operation directed to the bottom memory cell 1933, a data signal stored by the bottom memory cell 1933 selected by assertion of the BWL0 2026 propagates along the signal path 2510 onto the BBL 2024 (as a voltage signal), through the transistor 2012 activated by the bottom read word line signal BRWL, and onto the global bit line GBL as a current signal generated by the bottom gain cell 1935 on the RBL 2042. Stated another way, the bottom gain cell 1935 is configured to convert a voltage signal into a current signal. In some embodiments, the transistor 2052 remains deactivated by the top read word line signal TRWL during the read operation directed to the bottom memory cell 1933, such that the top gain cell 1945 is decoupled from the RBL 2042 and the read current on the RBL 2042 represents the data stored by the selected bottom memory cell 1933. In some embodiments, the current level developed on the RBL 2042 is determined by the data value stored by the selected bottom memory cell 1933. For example, when reading a logic low from the bottom memory cell 1933, the RBL 2042 and the global bit line GBL represent a low current level (IL), and when reading a logic high from the bottom memory cell 1933, the RBL 2042 and the global bit line GBL represent a high current level (IH).

Referring to FIG. 26, a diagram illustrating waveforms of signals, while reading the bottom memory cell 1933 through the RBL 2042, is shown, in accordance with some embodiments. For example, the bottom read word line signal BRWL, the signals present on the RBL 2042, the write enable signal WEN0, the write enable signal WEN1, and the top read word line signal TRWL are shown. It should be understood that the waveforms of FIG. 26 are provided merely for illustrative purposes, and do not intend to limit the scope of the present disclosure.

During a bottom bit line read operation, the bottom read word line signal BRWL transitions from a logic low voltage level (VL) to a logic high voltage level (VH) first, such that the transistor 2012 is activated to couple the bottom gain cell 1935 to the RBL 2042 and further to the global bit line GBL. After the bottom read word line signal BRWL transitions to VH, the RBL 2042 transitions from IL to IH according to the data value stored by the selected bottom memory cell 1933. The write enable signal WEN0, the write enable signal WEN1, and the top read word line signal TRWL are maintained at VL throughout the bottom bit line read operation, such that the transistor 2050, the transistor 2032, and the transistor 2052 remain deactivated, and the write bit line paths and the top read current path through the top gain cell 1945 are decoupled from the RBL 2042. After the bottom bit line read operation is completed, the bottom read word line signal BRWL transitions from VH back to VL. In some embodiments, the current level developed on the RBL 2042 is determined by the data value stored by the selected bottom memory cell 1933. For example, when reading a logic zero from the bottom memory cell 1933, the RBL 2042 and the global bit line GBL represent IL during the interval in which the bottom read word line signal BRWL is at VH, and when reading a logic one from the bottom memory cell 1933, the RBL 2042 and the global bit line GBL represent IH during the interval in which the bottom read word line signal BRWL is at VH.

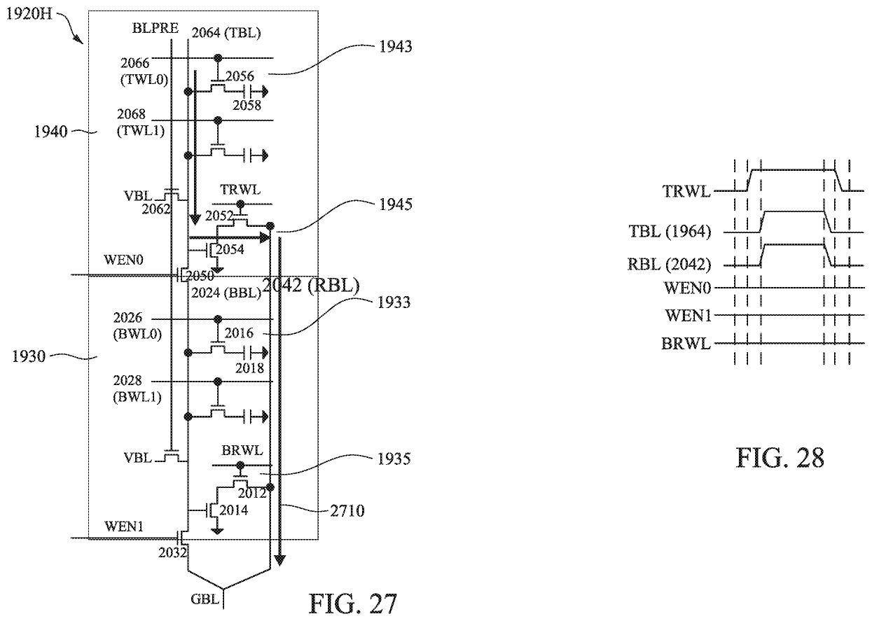

Referring to FIG. 27, the memory layer 1920H is shown as a representative example, with a signal path, shown as signal path 2710, directed to a read operation on the top memory cell 1943, in accordance with some embodiments. The RBL 2042 routes a far-side read path from the top gain cell 1945 to the global bit line GBL as one of the fly bit lines FBLs formed in a higher metallization layer, and is shared between the bottom array 1930 and the top array 1940 such that a one-bit read function directed to the top array 1940 is performed under control of the top read word line signal TRWL generated by the base die.

During a read operation directed to the top memory cell 1943, a data signal stored by the top memory cell 1943 selected by assertion of the TWL0 2066 propagates along the signal path 2710 onto the TBL 2064 (as a voltage signal), through the transistor 2052 activated by the top read word line signal TRWL, and onto the global bit line GBL as a current signal generated by the top gain cell 1945 on the RBL 2042. Stated another way, the top gain cell 1945 is configured to convert a voltage signal into a current signal. In some embodiments, the transistor 2012 remains deactivated by the bottom read word line signal BRWL during the read operation directed to the top memory cell 1943, such that the bottom gain cell 1935 is decoupled from the RBL 2042 and the read current on the RBL 2042 represents the data stored by the selected top memory cell 1943. In some embodiments, the current level developed on the RBL 2042 is determined by the data value stored by the selected top memory cell 1943. For example, when reading a logic low from the top memory cell 1943, the RBL 2042 and the global bit line GBL represent a low current level (IL), and when reading a logic high from the top memory cell 1943, the RBL 2042 and the global bit line GBL represent a high current level (IH).

Referring to FIG. 28, a diagram illustrating waveforms of signals, while reading the top memory cell 1943 through the RBL 2042, is shown, in accordance with some embodiments. For example, the top read word line signal TRWL, the signals present on the RBL 2042, the write enable signal WEN0, the write enable signal WEN1, and the bottom read word line signal BRWL are shown. It should be understood that the waveforms of FIG. 28 are provided merely for illustrative purposes, and do not intend to limit the scope of the present disclosure.

During a top bit line read operation, the top read word line signal TRWL transitions from a logic low voltage level (VL) to a logic high voltage level (VH) first, such that the transistor 2052 is activated to couple the top gain cell 1945 to the RBL 2042 and further to the global bit line GBL. After the top read word line signal TRWL transitions to VH, the RBL 2042 transitions from IL to IH according to the data value stored by the selected top memory cell 1943. The write enable signal WEN0, the write enable signal WEN1, and the bottom read word line signal BRWL are maintained at VL throughout the top bit line read operation, such that the transistor 2050, the transistor 2032, and the transistor 2012 remain deactivated, and the write bit line paths and the bottom read current path through the bottom gain cell 1935 are decoupled from the RBL 2042. After the top bit line read operation is completed, the top read word line signal TRWL transitions from VH back to VL. In some embodiments, the current level developed on the RBL 2042 is determined by the data value stored by the selected top memory cell 1943. For example, when reading a logic zero from the top memory cell 1943, the RBL 2042 and the global bit line GBL represent IL during the interval in which the top read word line signal TRWL is at VH, and when reading a logic one from the top memory cell 1943, the RBL 2042 and the global bit line GBL represent IH during the interval in which the top read word line signal TRWL is at VH.

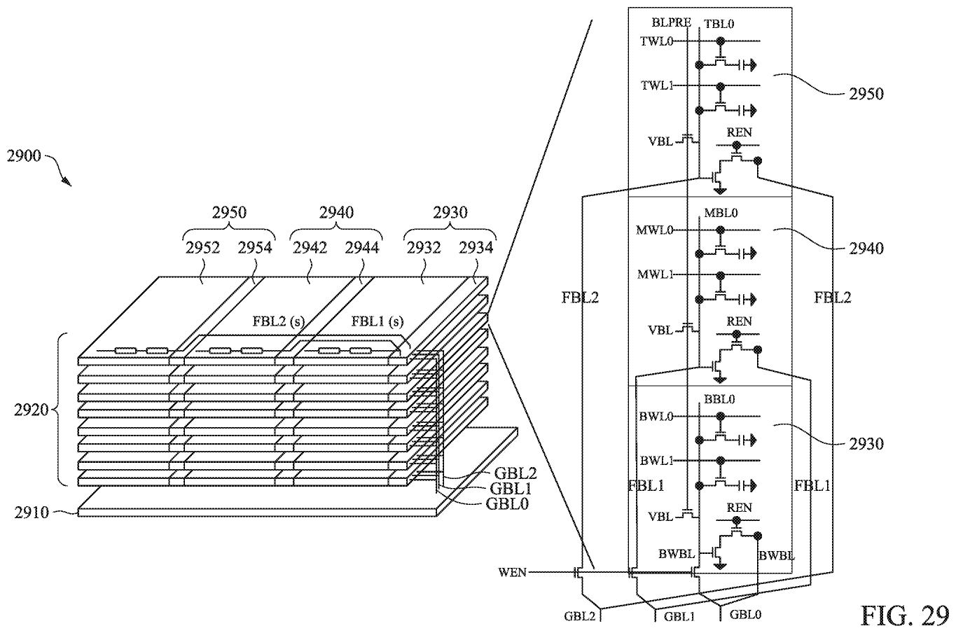

Referring to FIG. 29, a memory device 2900 can include a base die 2910 and a plurality of memory layers, shown as stacked memory arrays 2920, stacked over the base die 2910 along a vertical direction. The base die 2910 can include control, driver, and input/output (I/O) circuitry that facilitates read and write operations on the stacked memory arrays 2920. In some implementations, the stacked memory arrays 2920 can be respectively formed in a plurality of metallization layers disposed over a major surface of the base die 2910, such that the stacked memory arrays 2920 share the peripheral circuitry of the base die 2910. For example, the memory device 2900 can be arranged as an 8-stack memory or a 16-stack memory, with each of the stacked memory arrays 2920 providing a separate memory plane along the Z direction above the base die 2910 over the X-Y plane.

Each of the stacked memory arrays 2920 can include a plurality of array groups such as, for example, a bottom array 2930, a middle array 2940, and a top array 2950 disposed adjacent to one another along a lateral direction parallel to the lengthwise direction of local bit lines. The bottom array 2930 can include a bottom memory cell region 2932 and a bottom gain cell region 2934, the middle array 2940 can include a middle memory cell region 2942 and a middle gain cell region 2944, and the top array 2950 can include a plurality of top memory cells 2952 and a top gain cell region 2954. In some implementations, the bottom memory cell region 2932, the middle memory cell region 2942, and the top memory cells 2952 each include a plurality of memory cells each operable to store a data bit, and the bottom gain cell region 2934, the middle gain cell region 2944, and the top gain cell region 2954 each include a plurality of gain cells each operable to output a current representative of a stored logic state of a corresponding memory cell. In some embodiments, the bottom array 2930, the middle array 2940, and the top array 2950 may be disposed along the lateral direction such that the bottom array 2930 is a near-side group with respect to a set of global bit lines GBL0, GBL1, and GBL2, the middle array 2940 is a middle group, and the top array 2950 is a far-side group with respect to the global bit lines GBL0, GBL1, and GBL2.

The memory device 2900 can include the global bit line GBL0, the global bit line GBL1, and the global bit line GBL2 that vertically extend across the metallization layers in which the stacked memory arrays 2920 are formed. The global bit line GBL0, the global bit line GBL1, and the global bit line GBL2 can route read and write signals between the stacked memory arrays 2920 and the base die 2910. In some implementations, the global bit line GBL0 may be configured to route signals associated with the bottom array 2930 to the base die 2910, the global bit line GBL1 may be configured to route signals associated with the middle array 2940 to the base die 2910, and the global bit line GBL2 may be configured to route signals associated with the top array 2950 to the base die 2910. In some embodiments, a bottom write bit line BWBL and a bottom read bit line BRBL associated with the bottom array 2930 may be routed through a lower metallization layer as near-side bit lines coupled to the global bit line GBL0 through a transistor activated by a write enable signal WEN and a transistor activated by a read enable signal REN, respectively.

Each of the stacked memory arrays 2920 can include a plurality of fly bit lines such as, for example, a fly bit line FBL1 and a fly bit line FBL2 that respectively route a middle bit line path from the middle array 2940 across the bottom array 2930 to the global bit line GBL1 and a far-side bit line path from the top array 2950 across the middle array 2940 and the bottom array 2930 to the global bit line GBL2. The fly bit line FBL1 and the fly bit line FBL2 can be formed in a higher metallization layer than the local bit lines that couple the memory cells of the bottom memory cell region 2932, the middle memory cell region 2942, and the top memory cells 2952 along the lateral direction, such that the parasitic resistance and parasitic capacitance presented on the local bit lines are reduced. In some implementations, the fly bit line FBL1 extends laterally over the bottom memory cell region 2932 and the bottom gain cell region 2934 and couples to the global bit line GBL1, and the fly bit line FBL2 extends laterally over the bottom memory cell region 2932, the bottom gain cell region 2934, the middle memory cell region 2942, and the middle gain cell region 2944 and couples to the global bit line GBL2.

In some embodiments, the memory array of each of the stacked memory arrays 2920 may be divided into more than two groups, such as three groups as shown in FIG. 29, four groups, or n groups. For example, when the memory array is divided into n groups, the parasitic resistance and parasitic capacitance on the local bit line are reduced to approximately 1/n times the value associated with an undivided bit line of the same overall length, and the fly bit line approach is extended across the n groups such that a respective fly bit line routes a far-side bit line path from each of the n groups to a corresponding global bit line through a higher metallization layer.

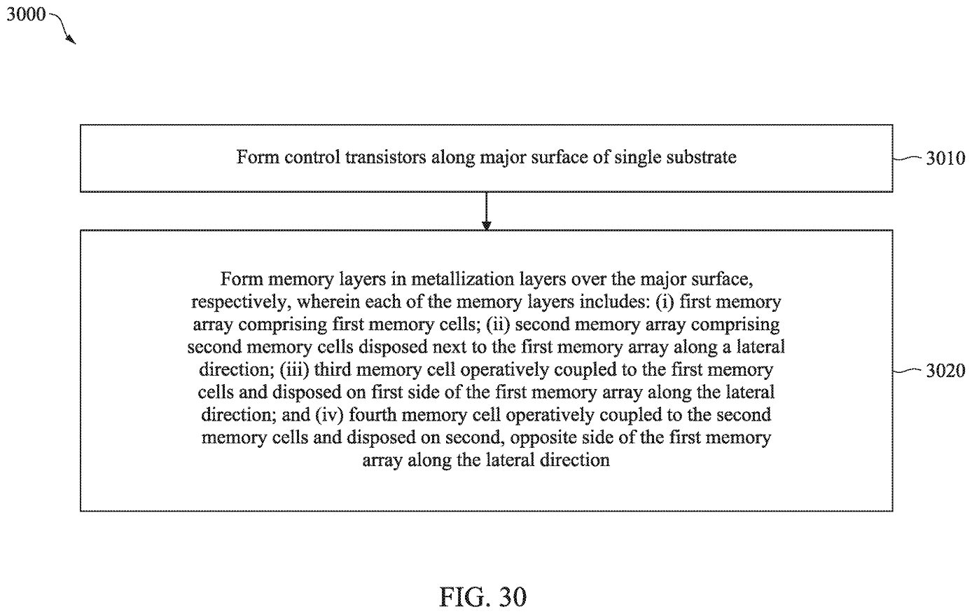

Referring to FIG. 30, a flow chart of an example method 3000 for fabricating a stacked memory device including a base die and a plurality of memory layers stacked thereover is shown, in accordance with some embodiments. For example, at least some of the operations (or steps) of the method 3000 can be used to fabricate the foregoing memory device (e.g., 100, 700, 1300, 1900, 2900). It should be appreciated that the method 3000 is merely an example, and does not intend to limit the scope of the present disclosure. Accordingly, it is understood that additional operations can be provided before, during, and after the method 3000 of FIG. 30, and that some other operations can only be briefly described herein.

The method 3000 begins with operation 3010 of forming a plurality of control transistors along a major surface of a single substrate. In some embodiments, the control transistors can collectively provide the control, driver, and input/output (I/O) circuitry formed on the base die described with respect to FIG. 1, FIG. 7, FIG. 13, FIG. 19, and FIG. 29, such that the control transistors facilitate read and write operations on the memory layers subsequently formed thereover. For example, the control transistors may be formed on a silicon substrate using a front-end-of-line (FEOL) process, and may include n-type transistors, p-type transistors, or a combination thereof arranged to generate various control signals such as, for example, the read enable signal REN, the write enable signal WEN, the write enable signal WEN0, the write enable signal WEN1, the top read word line signal TRWL, the bottom read word line signal BRWL, and/or the bit line pre-charge signal BLPRE.

The method 3000 continues to operation 3020 of forming memory layers in metallization layers over the major surface. In some embodiments, the memory layers are stacked along a vertical direction over the control transistors formed along the major surface of the single substrate, such that each of the memory layers is disposed in one or more respective ones of the metallization layers and shares the control transistors as common peripheral circuitry.

For example, the memory layers may be formed as 8 memory layers, 16 memory layers, or any suitable number of memory layer over the major surface using a back-end-of-line (BEOL) process. In various embodiments, as shown in FIG. 1, FIG. 7, FIG. 13, FIG. 19, and FIG. 29, each of the memory layers includes a first memory array (e.g., a bottom memory cell region) having a plurality of first memory cells, a second memory (e.g., a top memory cell region) array having a plurality of second memory cells disposed next to the first memory array along a lateral direction (e.g., the Y direction), a third memory cell (e.g. a bottom gain cell region) operatively coupled to the first memory cells and disposed on a first side of the first memory array along the lateral direction, and a fourth memory cell (e.g., a top gain cell region) operatively coupled to the second memory cells and disposed on a second, opposite side of the first memory array along the lateral direction.

In some embodiments, the operation 3020 can further include forming one or more fly bit lines (FBLs) in a higher metallization layer within each of the memory layers, such that a far-side bit line path extends laterally over the first memory array and couples to a global bit line vertically extending across the metallization layers. For example, the fly bit lines formed at the forming memory layers in metallization layers over the major surface may include a top read bit line (e.g., 242, 842, 1442), a top write bit line (e.g., 244), a shared read bit line (e.g., 2042), or a combination thereof arranged according to the configurations described with respect to FIG. 2, FIG. 8, FIG. 14, FIG. 20, and FIG. 29, such that the parasitic resistance and parasitic capacitance presented on the local bit lines of the memory layers are reduced relative to an arrangement in which both a near-side bit line path and a far-side bit line path are routed through a lower metallization layer.

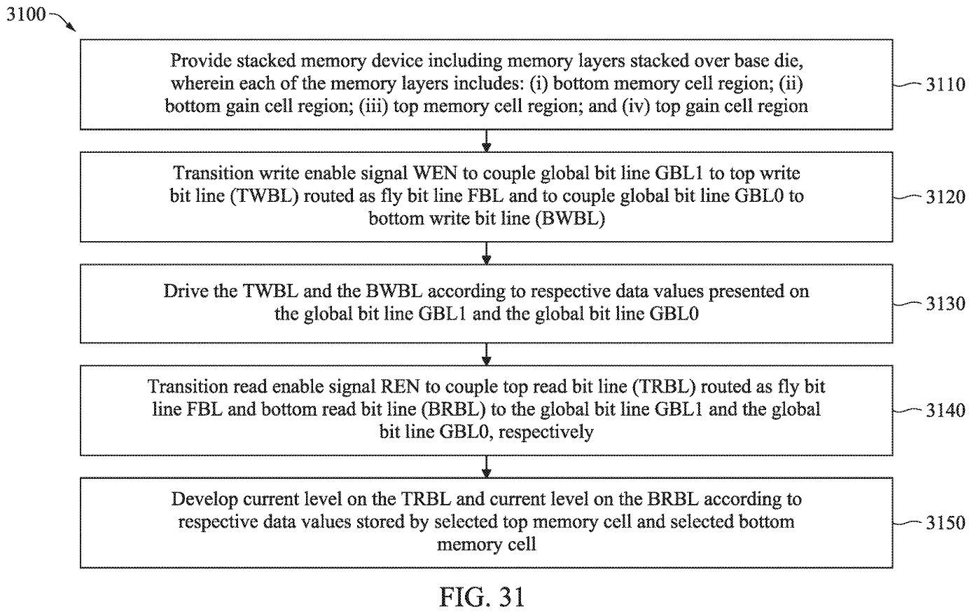

Referring to FIG. 31, a flow chart of an example method 3100 for operating a stacked memory device is disclosed. For example, the method 3100 can be utilized to operate the memory device 100 (FIG. 1). It should be appreciated that the method 3100 is merely an example, and does not intend to limit the scope of the present disclosure. Accordingly, it is understood that additional operations can be provided before, during, and after the method 3100 of FIG. 31, and that some other operations can only be briefly described herein.

The method 3100 can include operation 3110 of providing a stacked memory device including a plurality of memory layers stacked over a base die, in which each of the memory layers includes a bottom memory cell region, a bottom gain cell region, a top memory cell region, and a top gain cell region. In some implementations, the stacked memory device can include the memory device 100, with the base die 110 providing control, driver, and input/output circuitry, and the memory layer 120A through memory layer 120H stacked over the base die 110 along a vertical direction. Each of the memory layer 120A through memory layer 120H includes the bottom array 130 having the bottom memory cell region 132 and the bottom gain cell region 134, and the top array 140 having the top memory cell region 142 and the top gain cell region 144.

The method 3100 can include operation 3120 of transitioning a write enable signal WEN from a logic low voltage level to a logic high voltage level to couple a global bit line GBL1 to a top write bit line routed as a fly bit line FBL on a higher metallization layer and to couple a global bit line GBL0 to a bottom write bit line. For example, the transistor 272 and the transistor 232 can be activated by the write enable signal WEN to couple the global bit line GBL1 to the TWBL 244 and to couple the global bit line GBL0 to the BWBL 204, respectively.

The method can include operation 3130 of driving the TWBL 244 and the BWBL 204 according to respective data values presented on the global bit line GBL1 and the global bit line GBL0, such that a first data bit may be written to a top memory cell of the top memory cell region 142 along a signal path 320 and a second data bit may be written to a bottom memory cell of the bottom memory cell region 132 along a signal path 310 in parallel.

The method can include operation 3140 of transitioning a read enable signal REN from the logic low voltage level to the logic high voltage level to couple a top read bit line routed as a further fly bit line FBL on the higher metallization layer and a bottom read bit line to the global bit line GBL1 and the global bit line GBL0, respectively. For example, the transistor 252 and the transistor 212 can be activated by the read enable signal REN to couple the TRBL 242 to the global bit line GBL1 and to couple the BRBL 202 to the global bit line GBL0, respectively.

The method can include operation 3150 of developing a current level on the TRBL 242 and a current level on the BRBL 202 according to respective data values stored by a selected top memory cell accessed by assertion of the TWL0 266 and a selected bottom memory cell accessed by assertion of the BWL0 226, such that a first data bit may be read from the top memory cell region 142 along a signal path 520 and a second data bit may be read from the bottom memory cell region 132 along a signal path 510 in parallel.

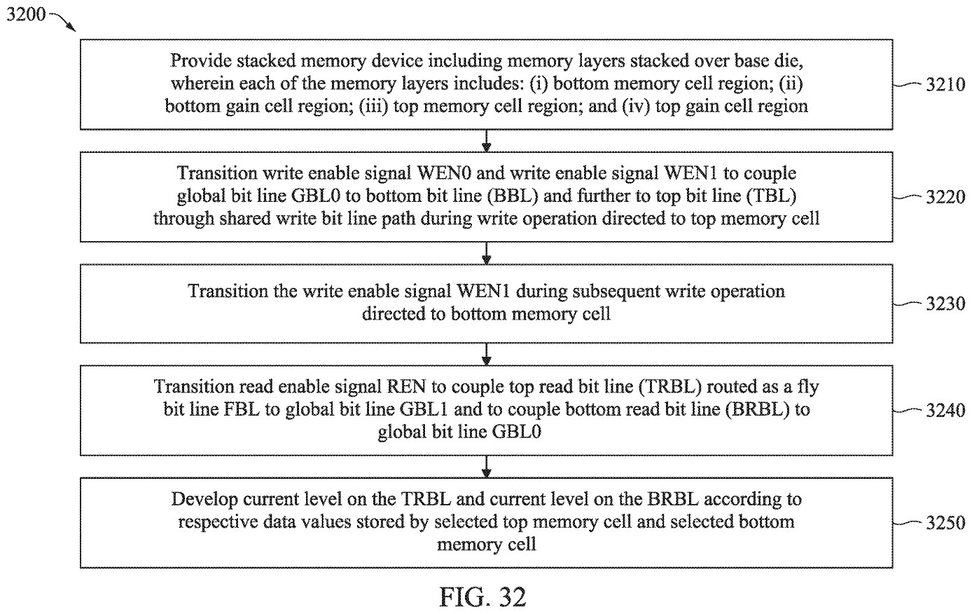

Referring to FIG. 32, a flow chart of an example method 3200 for operating a stacked memory device is disclosed. For example, the method 3200 can be utilized to operate the memory device 700 (FIG. 7). It should be appreciated that the method 3200 is merely an example, and does not intend to limit the scope of the present disclosure. Accordingly, it is understood that additional operations can be provided before, during, and after the method 3200 of FIG. 32, and that some other operations can only be briefly described herein.

The method 3200 can include operation 3210 of providing a stacked memory device including a plurality of memory layers stacked over a base die, in which each of the memory layers includes a bottom memory cell region, a bottom gain cell region, a top memory cell region, and a top gain cell region. In some implementations, the stacked memory device can include the memory device 700, with the base die 710 providing control, driver, and input/output circuitry, and the memory layer 720A through memory layer 720H stacked over the base die 710 along a vertical direction, in which each of the memory layer 720A through memory layer 720H includes the bottom array 730 having the bottom memory cell region 732 and the bottom gain cell region 734, and the top array 740 having the top memory cell region 742 and the top gain cell region 744.

The method 3200 can include operation 3220 of transitioning a write enable signal WEN0 and a write enable signal WEN1 from a logic low voltage level to a logic high voltage level to couple a global bit line GBL0 to a bottom bit line and further to a top bit line through a shared write bit line path during a top bit line write operation directed to the top memory cell 743. For example, the transistor 832 and the transistor 850 can be jointly activated to couple the global bit line GBL0 to the BBL 824 and further to the TBL 864, such that the BBL 824 and the TBL 864 transition from the logic low voltage level to the logic high voltage level according to a data value driven onto the global bit line GBL0.

The method 3200 can include operation 3230 of transitioning the write enable signal WEN1 from the logic low voltage level to the logic high voltage level while maintaining the write enable signal WEN0 at the logic low voltage level during a subsequent bottom bit line write operation directed to the bottom memory cell 733, such that the TBL 864 is decoupled from the BBL 824 and the top memory cell 743 is not disturbed by a next data value driven onto the global bit line GBL0. For example, the transistor 832 can be activated alone to couple the global bit line GBL0 to the BBL 824, such that the BBL 824 transitions from the logic low voltage level to the logic high voltage level according to the next data value, and the top memory cell 743 is written first and the bottom memory cell 733 is written thereafter using the shared write bit line path.

The method 3200 can include operation 3240 of transitioning a read enable signal REN from the logic low voltage level to the logic high voltage level to couple a top read bit line routed as a fly bit line FBL on a higher metallization layer to a global bit line GBL1 and to couple a bottom read bit line to the global bit line GBL0. For example, the transistor 852 and the transistor 812 can be activated by the read enable signal REN to couple the TRBL 842 to the global bit line GBL1 and to couple the BRBL 802 to the global bit line GBL0, respectively, while the write enable signal WEN0 and the write enable signal WEN1 are maintained at the logic low voltage level.

The method 3200 can include operation 3250 of developing a current level on the TRBL 842 and a current level on the BRBL 802 according to respective data values stored by a selected top memory cell accessed by assertion of the TWL0 866 and a selected bottom memory cell accessed by assertion of the BWL0 826, such that a first data bit may be read from the top memory cell 743 and a second data bit may be read from the bottom memory cell 733 in parallel.

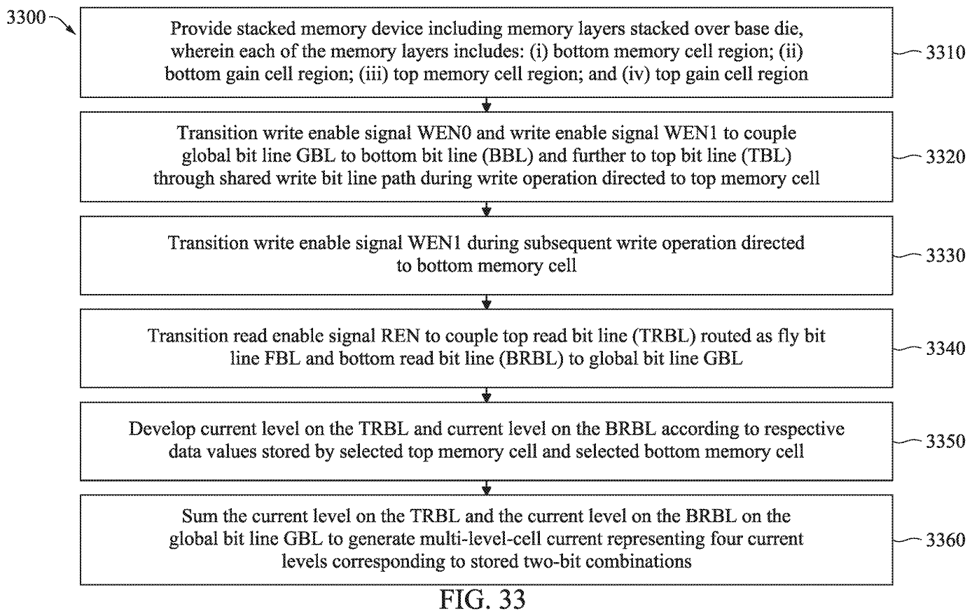

Referring to FIG. 33, a flow chart of an example method 3300 for operating a stacked memory device is disclosed. For example, the method 3300 can be utilized to operate the memory device 1300 (FIG. 13). It should be appreciated that the method 3300 is merely an example, and does not intend to limit the scope of the present disclosure. Accordingly, it is understood that additional operations can be provided before, during, and after the method 3300 of FIG. 33, and that some other operations can only be briefly described herein.

The method 3300 can include operation 3310 of providing a stacked memory device including a plurality of memory layers stacked over a base die, in which each of the memory layers includes a bottom memory cell region, a bottom gain cell region, a top memory cell region, and a top gain cell region. In some implementations, the stacked memory device can include the memory device 1300, with the base die 1310 providing control, driver, and input/output circuitry, and the memory layer 1320A through memory layer 1320H stacked over the base die 1310 along a vertical direction, in which each of the memory layer 1320A through memory layer 1320H includes the bottom array 1330 having the bottom memory cell region 1332 and the bottom gain cell region 1334, and the top array 1340 having the top memory cell region 1342 and the top gain cell region 1344.

The method 3300 can include operation 3320 of transitioning a write enable signal WEN0 and a write enable signal WEN1 from a logic low voltage level to a logic high voltage level to couple a global bit line GBL to a bottom bit line and further to a top bit line through a shared write bit line path during a top bit line write operation directed to the top memory cell 1343. For example, the transistor 1432 and the transistor 1450 can be jointly activated to couple the global bit line GBL to the BBL 1424 and further to the TBL 1464, such that the BBL 1424 and the TBL 1464 transition from the logic low voltage level to the logic high voltage level according to a data value driven onto the global bit line GBL.

The method 3300 can include operation 3330 of transitioning the write enable signal WEN1 from the logic low voltage level to the logic high voltage level while maintaining the write enable signal WEN0 at the logic low voltage level during a subsequent bottom bit line write operation directed to the bottom memory cell 1333, such that the TBL 1464 is decoupled from the BBL 1424 and the top memory cell 1343 is not disturbed by a next data value driven onto the global bit line GBL. For example, the transistor 1432 can be activated alone to couple the global bit line GBL to the BBL 1424, such that the BBL 1424 transitions from the logic low voltage level to the logic high voltage level according to the next data value, and the top memory cell 1343 is written first and the bottom memory cell 1333 is written thereafter using the shared write bit line path.

The method 3300 can include operation 3340 of transitioning a read enable signal REN from the logic low voltage level to the logic high voltage level to couple a top read bit line routed as a fly bit line FBL on a higher metallization layer and a bottom read bit line to the global bit line GBL, such that a current signal provided by the top gain cell 1345 is merged with a current signal provided by the bottom gain cell 1335 on the global bit line GBL. For example, the transistor 1452 and the transistor 1412 can be activated by the read enable signal REN to couple the TRBL 1442 and the BRBL 1402 to the global bit line GBL, while the write enable signal WEN0 and the write enable signal WEN1 are maintained at the logic low voltage level.

The method 3300 can include operation 3350 of developing a current level on the TRBL 1442 and a current level on the BRBL 1402 according to respective data values stored by a selected top memory cell accessed by assertion of the TWL0 1466 and a selected bottom memory cell accessed by assertion of the BWL0 1426, in which the top gain cell 1345 is sized larger than the bottom gain cell 1335 such that the current level on the TRBL 1442 is multiplied relative to the current level on the BRBL 1402. For example, when a size parameter M is equal to 2 for the top gain cell 1345 and equal to 1 for the bottom gain cell 1335, the TRBL 1442 transitions from a low current level to a doubled high current level when a logic high is read from the top memory cell 1343, and the BRBL 1402 transitions from the low current level to a high current level when a logic high is read from the bottom memory cell 1333.

The method 3300 can include operation 3360 of summing the current level on the TRBL 1442 and the current level on the BRBL 1402 on the global bit line GBL to generate a multi-level-cell current representing four current levels corresponding to stored two-bit combinations 00, 01, 10, and 11, such that the base die 1310 detects the four current levels on the global bit line GBL to distinguish the stored two-bit combinations.

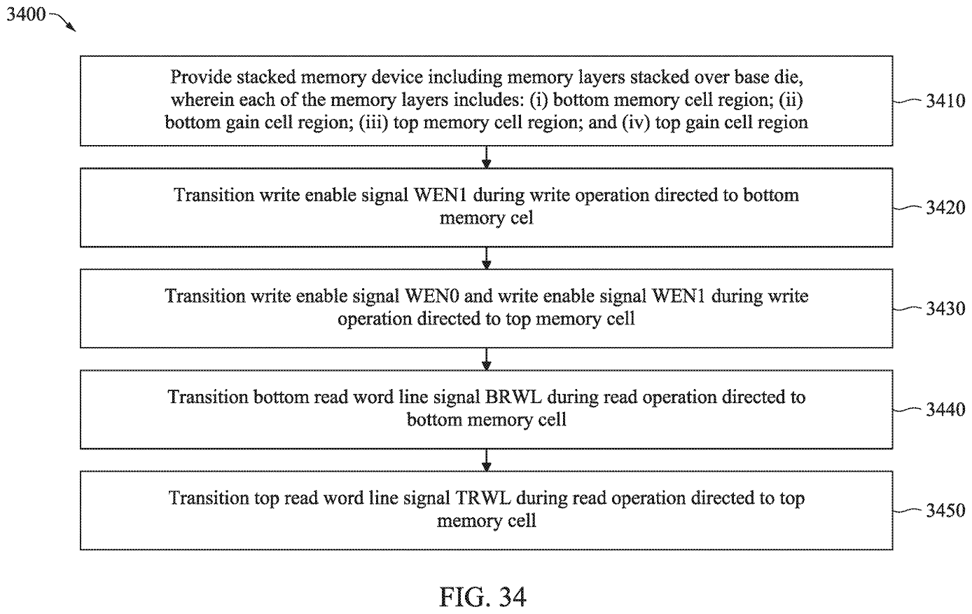

Referring to FIG. 34, a flow chart of an example method 3400 for operating a stacked memory device is disclosed. For example, the method 3400 can be utilized to operate the memory device 1900 (FIG. 19). It should be appreciated that the method 3400 is merely an example, and does not intend to limit the scope of the present disclosure. Accordingly, it is understood that additional operations can be provided before, during, and after the method 3400 of FIG. 34, and that some other operations can only be briefly described herein.

The method 3400 can include operation 3410 of providing a stacked memory device including a plurality of memory layers stacked over a base die, in which each of the memory layers includes a bottom memory cell region, a bottom gain cell region, a top memory cell region, and a top gain cell region. In some implementations, the stacked memory device can include the memory device 1900, with the base die 1910 providing control, driver, and input/output circuitry, and the memory layer 1920A through memory layer 1920H stacked over the base die 1910 along a vertical direction, in which each of the memory layer 1920A through memory layer 1920H includes the bottom array 1930 having the bottom memory cell region 1932 and the bottom gain cell region 1934, and the top array 1940 having the top memory cell region 1942 and the top gain cell region 1944.

The method 3400 can include operation 3420 of transitioning a write enable signal WEN1 from a logic low voltage level to a logic high voltage level while maintaining a write enable signal WEN0 at the logic low voltage level during a bottom bit line write operation directed to the bottom memory cell 1933, such that the TBL 2064 is decoupled from the BBL 2024 and the top memory cell 1943 is not disturbed by a data value driven onto the global bit line GBL. For example, the transistor 2032 can be activated alone to couple the global bit line GBL to the BBL 2024, such that the BBL 2024 transitions from the logic low voltage level to the logic high voltage level according to the data value, and the capacitor 2018 of the selected bottom memory cell 1933 is charged or discharged through the transistor 2016 activated by assertion of the BWL0 2026.

The method 3400 can include operation 3430 of transitioning both the write enable signal WEN0 and the write enable signal WEN1 from the logic low voltage level to the logic high voltage level during a top bit line write operation directed to the top memory cell 1943, such that the transistor 2032 and the transistor 2050 are jointly activated to couple the global bit line GBL to the BBL 2024 and further to the TBL 2064. For example, the BBL 2024 and the TBL 2064 can transition from the logic low voltage level to the logic high voltage level according to a next data value driven onto the global bit line GBL, such that the capacitor 2058 of the selected top memory cell 1943 is charged or discharged through the transistor 2056 activated by assertion of the TWL0 2066, while a bottom read word line signal BRWL and a top read word line signal TRWL are maintained at the logic low voltage level.

The method 3400 can include operation 3440 of transitioning the bottom read word line signal BRWL from the logic low voltage level to the logic high voltage level during a bottom bit line read operation directed to the bottom memory cell 1933, such that the transistor 2012 is activated to couple the bottom gain cell 1935 to the RBL 2042 and further to the global bit line GBL, while the write enable signal WEN0, the write enable signal WEN1, and the top read word line signal TRWL are maintained at the logic low voltage level. For example, the RBL 2042 can transition from a low current level to a high current level according to a data value stored by a selected bottom memory cell accessed by assertion of the BWL0 2026, such that the current level developed on the RBL 2042 represents the data value stored by the selected bottom memory cell 1933.

The method 3400 can include operation 3450 of transitioning the top read word line signal TRWL from the logic low voltage level to the logic high voltage level during a top bit line read operation directed to the top memory cell 1943, such that the transistor 2052 is activated to couple the top gain cell 1945 to the RBL 2042 and further to the global bit line GBL, while the write enable signal WEN0, the write enable signal WEN1, and the bottom read word line signal BRWL are maintained at the logic low voltage level. For example, the RBL 2042 can transition from the low current level to the high current level according to a data value stored by a selected top memory cell accessed by assertion of the TWL0 2066, such that the RBL 2042 is reused as a shared read bit line path for the top array 1940 and the bottom array 1930 under selective control of the top read word line signal TRWL and the bottom read word line signal BRWL generated by the base die 1910.

In one aspect of the present disclosure, a device is disclosed. The device can include a plurality of control transistors formed along a major surface of a single substrate. The device can include a plurality of memory layers respectively formed in a plurality of metallization layers that are disposed over the major surface. Each of the memory layers can include a first memory array comprising a plurality of first memory cells. Each of the memory layers can include a second memory array comprising a plurality of second memory cells. Each of the memory layers can include a third memory cell operatively coupling the plurality of control transistors to the plurality of first memory cells. Each of the memory layers can include a fourth memory cell operatively coupling the plurality of control transistors to the plurality of second memory cells.

In another aspect of the present disclosure, a device is disclosed. The device can include a first memory layer. The first memory layer can include a first memory array comprising a plurality of first memory cells. The first memory layer can include a second memory array comprising a plurality of second memory cells. The first memory layer can include a third memory cell operatively coupled to the first memory cells. The first memory layer can include a fourth memory cell operatively coupled to the plurality of second memory cells. The first memory array and the second memory array can be disposed adjacent to each other along a lateral direction, with the fourth memory cell interposed therebetween along the lateral direction and with the third memory cell disposed opposite the first memory array from the fourth memory cell along the lateral direction. The third memory cell can be configured to convert a first voltage signal generated by at least one of the first memory cells into a first current signal. The fourth memory cell can be configured to convert a second voltage signal generated by at least one of the second memory cells into a second current signal.

In yet another aspect of the present disclosure, a method is disclosed. The method can include forming a plurality of control transistors along a major surface of a single substrate. The method can include forming a plurality of memory layers in a plurality of metallization layers over the major surface, respectively. Each of the plurality of memory layers can include a first memory array comprising a plurality of first memory cells. Each of the plurality of memory layers can include a second memory array comprising a plurality of second memory cells disposed next to the first memory array along a lateral direction. Each of the plurality of memory layers can include a third memory cell operatively coupled to the first memory cells and disposed on a first side of the first memory array along the lateral direction. Each of the plurality of memory layers can include a fourth memory cell operatively coupled to the plurality of second memory cells and disposed on a second, opposite side of the first memory array along the lateral direction.

As used herein, the terms “about” and “approximately” generally indicates the value of a given quantity that can vary based on a particular technology node associated with the subject semiconductor device. Based on the particular technology node, the term “about” can indicate a value of a given quantity that varies within, for example, 10-30% of the value (e.g., ±10%, ±20%, or ±30% of the value).

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A device, comprising:
a plurality of control transistors formed along a major surface of a single substrate; and
a plurality of memory layers respectively formed in a plurality of metallization layers that are disposed over the major surface;
wherein each of the memory layers comprises at least: (i) a first memory array comprising a plurality of first memory cells; (ii) a second memory array comprising a plurality of second memory cells; (iii) a third memory cell operatively coupling the plurality of control transistor to the plurality of first memory cells; and (iv) a fourth memory cell operatively coupling the plurality of control transistor to the plurality of second memory cells.

2. The device of claim 1, wherein the plurality of first memory cells each include a first transistor and a first capacitor, the plurality of second memory cells each include a second transistor and a second capacitor, the third memory cell includes a pair of third transistors, and the fourth memory cell includes a pair of fourth transistors.

3. The device of claim 1, wherein the plurality of first memory cells are physically arranged along a lateral direction, the plurality of second memory cells are physically arranged along the lateral direction, the third memory cell is arranged on a first side of the first memory array along the lateral direction, the fourth memory cell is arranged on a second, opposite side of the first memory array along the lateral direction and interposed between the first and second memory arrays along the lateral direction.

4. The device of claim 1, further comprising:
a first bit line connected to a source/drain terminal of one transistor of the third memory cell;
a second bit line connected to a gate terminal of another transistor of the third memory cell and respective source/drain terminals of transistors of the first memory cells;
a third bit line connected to a source/drain terminal of one transistor of the fourth memory cell; and
a fourth bit line connected to a gate terminal of another transistor of the fourth memory cell and respective source/drain terminals of transistors of the second memory cells;
wherein the second bit line and the fourth bit line are electrically decoupled from each other.

5. The device of claim 4, further comprising:
a first global bit line vertically extending across the plurality of metallization layers, and connected to the first bit line and the second bit line; and
a second global bit line vertically extending across the plurality of metallization layers, and connected to the third bit line and the fourth bit line.

6. The device of claim 4, wherein the third bit line and the fourth bit line each extend over the first memory array.

7. The device of claim 1, further comprising:
a first bit line connected to a source/drain terminal of one transistor of the third memory cell;
a first portion of a second bit line connected to a gate terminal of another transistor of the third memory cell and respective source/drain terminals of transistors of the first memory cells;
a third bit line connected to a source/drain terminal of one transistor of the fourth memory cell; and
a second portion of the second bit line connected to a gate terminal of another transistor of the fourth memory cell and respective source/drain terminals of transistors of the second memory cells.

8. The device of claim 7, further comprising:
a first global bit line vertically extending across the plurality of metallization layers, and connected to the first bit line and the second bit line; and
a second global bit line vertically extending across the plurality of metallization layers, and connected to the third bit line.

9. The device of claim 7, wherein the third bit line extends over the first memory array.

10. The device of claim 1, further comprising:
a first bit line connected to a source/drain terminal of one transistor of the third memory cell and a source/drain terminal of one transistor of the fourth memory cell;
a first portion of a second bit line connected to a gate terminal of another transistor of the third memory cell and respective source/drain terminals of transistors of the first memory cells; and
a second portion of the second bit line connected to a gate terminal of another transistor of the fourth memory cell and respective source/drain terminals of transistors of the second memory cells.

11. The device of claim 10, further comprising:
a global bit line vertically extending across the plurality of metallization layers, and connected to the first bit line and the second bit line.

12. The device of claim 10, wherein the first bit line includes a portion extending over the first memory array.

13. A device, comprising:
a first memory layer comprising at least: (i) a first memory array comprising a plurality of first memory cells; (ii) a second memory array comprising a plurality of second memory cells; (iii) a third memory cell operatively coupled to the first memory cells; and (iv) a fourth memory cell operatively coupled to the plurality of second memory cells;
wherein the first memory array and the second memory array are disposed adjacent to each other along a lateral direction, with the fourth memory cell interposed therebetween along the lateral direction and with the third memory cell disposed opposite the first memory array from the fourth memory cell along the lateral direction;
wherein the third memory cell is configured to convert a first voltage signal generated by at least one of the first memory cells into a first current signal; and
wherein the fourth memory cell is configured to convert a second voltage signal generated by at least one of the second memory cells into a second current signal.

14. The device of claim 13, further comprising:
a second memory layer comprising at least: (i) a fifth memory array comprising a plurality of fifth memory cells; (ii) a sixth memory array comprising a plurality of sixth memory cells; (iii) a seventh memory cell operatively coupled to the fifth memory cells; and (iv) an eighth memory cell operatively coupled to the plurality of sixth memory cells;
wherein the fifth memory array and the sixth memory array are disposed adjacent to each other along the lateral direction, with the eighth memory cell interposed therebetween along the lateral direction and with the seventh memory cell disposed opposite the fifth memory array from the eighth memory cell along the lateral direction;
wherein the seventh memory cell is configured to convert a third voltage signal generated by at least one of the fifth memory cells into a third current signal;
wherein the eighth memory cell is configured to convert a fourth voltage signal generated by at least one of the sixth memory cells into a fourth current signal; and
wherein the first memory layer and the second memory layer are vertically spaced from each other.

15. The device of claim 14, further comprising:
a plurality of control transistors formed along a major surface of a single substrate;
wherein the first memory layer and the second memory layer are vertically disposed over the major surface.

16. The device of claim 13, wherein the plurality of first memory cells each include a first transistor and a first capacitor, the plurality of second memory cells each include a second transistor and a second capacitor, the third memory cell includes a pair of third transistors, and the fourth memory cell includes a pair of fourth transistors.

17. The device of claim 13, further comprising:
a first bit line connected to a source/drain terminal of one transistor of the third memory cell;
a second bit line connected to a gate terminal of another transistor of the third memory cell and respective source/drain terminals of transistors of the first memory cells;
a third bit line connected to a source/drain terminal of one transistor of the fourth memory cell; and
a fourth bit line connected to a gate terminal of another transistor of the fourth memory cell and respective source/drain terminals of transistors of the second memory cells.

18. The device of claim 13, further comprising:
a first bit line connected to a source/drain terminal of one transistor of the third memory cell;
a first portion of a second bit line connected to a gate terminal of another transistor of the third memory cell and respective source/drain terminals of transistors of the first memory cells;
a third bit line connected to a source/drain terminal of one transistor of the fourth memory cell; and
a second portion of the second bit line connected to a gate terminal of another transistor of the fourth memory cell and respective source/drain terminals of transistors of the second memory cells.

19. A method, comprising:
forming a plurality of control transistors along a major surface of a single substrate; and
forming a plurality of memory layers in a plurality of metallization layers over the major surface, respectively;
wherein each of the plurality of memory layers includes: (i) a first memory array comprising a plurality of first memory cells; (ii) a second memory array comprising a plurality of second memory cells disposed next to the first memory array along a lateral direction; (iii) a third memory cell operatively coupled to the first memory cells and disposed on a first side of the first memory array along the lateral direction; and (iv) a fourth memory cell operatively coupled to the plurality of second memory cells and disposed on a second, opposite side of the first memory array along the lateral direction.

20. The method of claim 19, wherein the plurality of first memory cells each include a first transistor and a first capacitor, the plurality of second memory cells each include a second transistor and a second capacitor, the third memory cell includes a pair of third transistors, and the fourth memory cell includes a pair of fourth transistors.
